(19) United States
(12) Patent Application Publication
(10) Pub. No.: US 2026/0307129 A1
(43) Pub. Date: Oct. 8, 2026

(54) SYSTEMS, METHODS, AND DEVICES FOR AUTOMATED BENDING OF FIXATION PLATES

(71) Applicant: Ohio State Innovation Foundation, Clumbus, OH (US)

(72) Inventors: Brian THURSTON, Columbus, OH (US); H. David DEAN, Upper Arlington, OH (US); Javier Oswaldo VAZQUEZ ARMENDARIZ, Columbus, OH (US); Glenn DAEHN, Columbus, OH (US); Stephen NIEZGODA, Worthington, OH (US); Michael GROEBER, Columbus, OH (US); Luis Hector OLIVAS ALANIS, Columbus, OH (US)

(21) Appl. No.: 19/479,165

(22) PCT Filed: Apr. 29, 2024
(86) PCT No.: PCT/US2024/026865

§ 371 Date: Oct. 27, 2025

Related U.S. Application Data
(60) Provisional application No. 63/462,838, filed on Apr. 28, 2023.
(60) Provisional application No. 63/500,432, filed on May 5, 2023.

(51) Int. Cl. B21D 7/12; A61B 17/88; B21D 7/06

(52) U.S. Cl. CPC B21D 7/12; A61B 17/8863; B21D 7/06

(57) ABSTRACT

A device for bending a fixation plate may include two pairs of opposing jaws, each including two members disposed on opposite sides of a bending area. Each of the four members includes a tip configured to engage with a tip of an opposing member. The members of the pairs of jaws extend away from the bending area at an angle such that one or more clearance areas are defined between opposing members. The device may include at least one actuator configured to move a first pair of jaws between (i) an engage configuration wherein a first tip engages a second tip and (ii) a separated configuration wherein the first tip is separated from the second tip by a distance. The at least one actuator is further configured to rotate a second pair of jaws to an angled configuration with respect to the bending area.

### CROSS-REFERENCE TO RELATED APPLICATIONS

This application claims priority to U.S. Provisional Patent Application No. 63/462,838, filed Apr. 28, 2023, and U.S. Provisional Patent Application No. 63/500,432, filed May 5, 2023, each of which is incorporated herein by reference in its entirety.

### STATEMENT REGARDING FEDERALLY SPONSORED RESEARCH OR DEVELOPMENT

This invention was made with government support under grant no. 2133630 awarded by the National Science Foundation. The government has certain rights in the invention.

### BACKGROUND

The present disclosure generally relates to Point-of-Care Manufacturing (POCM) of medical devices. Currently, POCM services usually begin with the need for personalization of medical devices for a patient's medical condition. 3D Computed Tomography (CT) or 3D Magnetic Resonance Imaging (MRI) scans are obtained for diagnostic purposes and then used to create 3D anatomic models. Following segmentation of the anatomy of interest, the isolated 3D surfaces are then used in a Virtual Surgical Planning (VSP) environment. VSP is the process of taking patient-specific data to design a surgical intervention and often to create 3D models of a surgical reconstruction. Those surgical models can be 3D printed for use in planning and as an intra-operative reference. Additionally, the surgical plan created in VSP software can be employed in stereotactic intra-operative image guidance systems. These strategies have demonstrated many benefits including Operating Room (OR) time reduction, improving surgical outcomes, and decreasing hardware failure rates.

Despite recent advances in Point-of-Care Manufacturing (POCM) and Virtual Surgical Planning (VSP) strategies for medical device manufacturing, there are still gaps in most design engineering, decision-making based on expected mechanical performance, selection of materials, and fabrication workflows, including for craniomaxillofacial (CMF) fixation hardware (e.g., fixation plates) to treat trauma, cancer, or other rapidly emerging serious conditions. Some areas of opportunity are the limited shape variation of hardware provided by vendors, which is approved based on medical judgment rather than a mechanical optimization report for choice of material, shape, fixation hardware location, and screw depth and location. Therefore, the lack of design and personalization of these devices with optimized function and fit might compromise their performance.

Therefore, there is a need for improved systems, methods, and devices for point-of-care manufacturing of medical devices.

### SUMMARY

One implementation of the present disclosure is a bending device including a first pair of jaws, a second pair of jaws, and at least one actuator. The first pair of opposing jaws on a first end of the device includes a first member disposed on a first side of a bending area and a corresponding second member disposed on a second side of the bending area opposite of the first side. The first and second members extend away from the bending area at an angle such that a first clearance area is defined between the first and second members. A first tip of the first member is configured to engage with a second tip of the second member within the bending area. The second pair of opposing jaws on a second end of the device opposite the first end includes a third member disposed on the first side of the bending area and a corresponding fourth member disposed on the second side of the bending area opposite of the first side. The third and fourth members extend away from the bending area at an angle such that a second clearance area is defined between the third and fourth members. A third tip of the third member is configured to engage with a fourth tip of the fourth member within the bending area. The at least one actuator is configured to move the first pair of jaws between (i) an engaged configuration wherein the first tip engages the second tip and (ii) a separated configuration wherein the first tip is separated from the second tip by a distance. The at least one actuator is further configured to rotate the second pair of jaws to an angled configuration with respect to the bending area.

In some implementations, the at least one actuator is further configured to move the second pair of jaws between (i) an engaged configuration wherein the third tip engages the fourth tip and (ii) a separated configuration wherein the third tip is separated from the fourth tip by a distance, the at least one actuator further configured to rotate the first pair of jaws to the angled configuration.

In some implementations, the first and third members extend away from the bending area such that a third clearance area is defined between the first and third members, and wherein the second and fourth members extend away from the bending area such that a fourth clearance area is defined between the second and fourth members.

In some implementations, the second pair of jaws are moved to the angled configuration, the third member moves into the third clearance area closer to the first member.

In some implementations, the third and fourth clearance areas ensure that the first pair of jaws and the second pair of jaws do not interfere with each other during operation of the bending device.

In some implementations, rotation of the second pair of jaws to the angled configuration includes one or more of: (i) rotation of the second pair of jaws about a first axis, defined as a roll rotation; (ii) rotation of the second pair of jaws about a second axis, defined as a pitch rotation; and (iii) rotation of the second pair of jaws about a third axis, defined as a yaw rotation.

In some implementations, the bending area includes a center point of rotation defined in between each of the first, second, third, and fourth tips.

In some implementations, the first tip of the first member of the first pair of opposing jaws includes a protrusion and the second tip of the second member of the first pair of opposing jaws includes a cavity, wherein the protrusion engages the cavity when the first pair of opposing jaws is in the engaged configuration.

In some implementations, the bending device further includes a controller in communication with and configured to activate the at least one actuator to move one or more of the first pair of opposing jaws and the second pair of opposing jaws.

In some implementations, the controller includes a set of manufacturing process instructions for moving the first and second pair of opposing jaws in a predefined sequence.

In some implementations, the instructions are based on a computational model of a patient's anatomy or a model of an implant.

In some implementations, the second pair of opposing jaws is configured to bend a bone fixation plate to a desired angle or an angled configuration.

Another implementation of the present disclosure is a system including a bending device, at least one actuator, a medical implant, and a control system. The bending device includes a first pair of opposing jaws including a first member disposed on a first side of a bending area and a corresponding second member disposed on a second side of the bending area opposite of the first side. The first and second members extend away from the bending area at an angle such that a first clearance area is defined between the first and second members. A first tip of the first member is configured to engage with a second tip of the second member within the bending area. A second pair of opposing jaws includes a third member disposed on the first side of the bending area and a corresponding fourth member disposed on the second side of the bending area opposite of the first side. The third and fourth members extend away from the bending area at an angle such that a second clearance area is defined between the third and fourth members. A third tip of the third member is configured to engage with a fourth tip of the fourth member within the bending area. The at least one actuator is coupled to the bending device and configured to move the first pair of jaws between (i) an engaged configuration wherein the first tip engages the second tip and (ii) a separated configuration wherein the first tip is separated from the second tip by a distance. The at least one actuator is further configured to rotate the second pair of jaws to an angled configuration with respect to the bending area. The medical implant is disposed within the bending area, and each of the first pair of opposing jaws and the second pair of opposing jaws are configured to engage with the medical implant. The control system is in communication with the bending device, the control system including computer readable instructions for bending the medical implant to a desired shape, the computer readable instructions being based on an anatomical model of a patient.

In some implementations, the medical implant is a bone fixation plate.

In some implementations, the bone fixation plate includes a plurality of eyelets and wherein the first and second tip of the first pair of jaws are used to index or locate the bone fixation plate via the plurality of eyelets.

Another implementation of the present disclosure is a method of shaping a medical implant. The method includes: providing a device including a first pair of opposing jaws on an input side of a bending area and a second pair of opposing jaws on an output side of a bending area, wherein each of the first and second pair of opposing jaws further define one or more clearance zones to prevent interference between the first and second pairs of opposing jaws; moving the medical implant into the bending area between the first pair of opposing jaws from the input side; closing, via at least one actuator, the first pair of opposing jaws to engage the medical implant at a first portion of the medical implant; closing, via the at least one actuator, the second pair of opposing jaws to engage the medical implant at a second portion of the medical implant; and turning, via the least one actuator, the second pair of opposing jaws to a first angled configuration with respect to the bending area such that the second portion of the medical implant is angled with respect to the first portion of the medical implant.

In some implementations, the method further includes opening, via the at least one actuator, the first pair of opposing jaws; and moving the first pair of opposing jaws to a third portion of the medical implant that is closer to the input side than the first portion of the medical implant and closing the first pair of opposing jaws to engage the third portion of the medical implant.

In some implementations, the method further includes: opening, via the at least one actuator, the second pair of opposing jaws; and moving the second pair of opposing jaws to the second portion of the medical implant and closing the second pair of opposing jaws to engage the third portion of the medical implant.

In some implementations, the medical implant is shaped into a custom topology via one or more steps of turning the second pair of opposing jaws to one or more angled configurations, wherein the custom topology is based on a model of a patient's anatomy.

In some implementations, in each of the one or more steps of turning the second pair of opposing jaws, the medical implant experiences a single bending operation, reducing iterative bending of a single area that may produce fatigue.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 shows a diagram of Hybrid Autonomous Manufacturing (HAM) including Point-of-Care Manufacturing (POCM) of a hip implant via robotic machining with sensors (upper), and a schematic of POCM HAM robots near OR (lower), according to one implementation.

FIG. 2 shows a diagram of a robotic Point-of-Care Manufacturing (POCM) workflow, according to one implementation.

FIG. 3 shows a workflow for designing a specific-patient mandibular fixation plate, according to one implementation.

FIG. 4 shows a diagram with the stages of a plate fabrication process, according to one implementation.

FIG. 5 shows a model of a bone fixation plate on a mandible with gaps between the fixation plate, according to one implementation.

FIG. 6 provides the typical shape of a fixation plate, according to one implementation.

FIG. 7 provides a fixation plate having branches, according to one implementation.

FIG. 8 shows a device for bending a medical device, according to one implementation.

FIG. 9 shows a side view of the device of FIG. 8 wherein each pair of jaws is engaged with an eyelet of the bone fixation plate, according to one implementation.

FIG. 10 shows a side view of the device wherein the second pair of jaws have been rotated to bend the fixation plate, according to one implementation.

FIGS. 11A-11I show a sequence of steps for performing a bending operation and subsequent bending operations using the disclosed device, according to various implementations.

FIGS. 12A-12C show the device of FIG. 8 with the second pair of jaws performing a rotation, according to one implementation.

FIGS. 13A-13C show the device of FIG. 8 with the second pair of jaws performing a rotation, according to another implementation.

FIGS. 14A-14C show the members of the pairs of jaws of an example device at various clearance angles concept, according to one implementation.

FIGS. 15A-15C show another implementation of the disclosed device to demonstrate the clamping jaw thickness, according to one implementation.

FIG. 16 illustrates a bent bone fixation device extending into the space on the product side of the device, according to one implementation.

FIG. 17 shows an implementation of an indexing member of the disclosed device, according to one implementation.

FIG. 18 shows another implementation of an indexing member of the disclosed device, according to another implementation.

FIG. 19 shows another implementation of an indexing member of the disclosed device, according to another implementation.

FIGS. 20 and 21 a system and device for bending a bone fixation plate, along with associated framing, actuators, and controllers, according to one implementation.

FIGS. 22-24 show another implementation of a system and device for bending a fixation plate, according to one implementation.

FIGS. 25-29 show another implementation system and device for bending a bone fixation plate (e.g., a plate bending machine), according to one implementation.

FIG. 30 shows images of finished bone fixation plates formed by the devices of this disclosure, according to one implementation.

FIG. 31 shows various skull models including surface curvature-mapped 3D CT images used to create wireframe skull templates, according to one implementation.

FIG. 32 shows a homology map used to superimpose a normative biomechanical model of chewing to a patient's 3D CT-based image, along with stress concentration maps for specific fixation plates, according to one implementation.

FIG. 33 shows a POCM design envelope with an optimized fabrication and device function, according to one implementation.

FIG. 34 shows a diagram of the design and creation of plates for surgical re-enactment on biomimetic models, according to one implementation.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, systems, methods, and devices for medical device manufacturing, including point-of-care manufacturing (POCM) are shown, according to various implementations.

### Clinical Background

Also missing in current POCM strategies is the identification of the anticipated physiological load, such as chewing forces for the maxilla or mandible. In addition, in most cases it is neglected the use of those loading regimes to determine the optimal position of the hardware so that it has appropriate strength to accomplish these primary goals: (1) design and testing the fixation so that grafted bone is only under compression (i.e., to bear all loading during healing) avoiding wound healing site damage by placing healing sites under tension, (2) reducing micromotion during healing to lower the risk of failed bone healing and insufficient vascularization, and (3) ensuring that the wound healing fixation does not interrupt normal stress-strain trajectories of the healed bone.

An approach to unify such design and manufacturing efforts could be achieved by means of Integrated Computational Materials Engineering (ICME), where the design of a product is based on its manufacturing processes, material microstructure, and engineering properties at different length scales. Its applicability, for example, has proven to be beneficial in the prediction of microstructure evolution in metals during hot rolling processes. By using different design variables and process parameters during modeling, great flexibility can be obtained in the tailoring of material properties, and thus, subsequently, in the performance of fabricated parts. The same concept could be adapted to develop a methodology that considers relevant information from the fabrication process of skeletal fixation plates to anticipate changes in their microstructure, as well as input data on the patient's chewing biomechanics for personalization. By also incorporating the use of Metamorphic Manufacturing (MM) as an approach to rely on closed-loop forming methodologies and Hybrid Autonomous Manufacturing (HAM) to bring together materials and processes with sensing, Artificial Intelligence (AI) and Machine Learning (ML), personalized fabrication of fixation plates with the required quality and desired engineering properties for optimal performance would be supported. With these approaches, the medical device design and manufacturing systems would provide the surgeon with a computational graphical rendering of how critical choices in device materials and geometry will affect the performance and durability of the component, post-healing, and how the period of highest restored function might be achieved earlier and sustained longer. The ICME framework integrates length scales and processes to performance. In a very simple example, it is well known that residual stress is a dominant factor in component performance with respect to fatigue and stress corrosion in susceptible materials. Further, the detailed sequence of bending operations will affect the distribution and magnitudes of residual stresses. Simple ICME-inspired calculations may locate compressive residual stresses at locations of maximum repeated tensile stress to improve component performance. At a more ambitious level, modeling implant and biological materials in a full rendering-planning-tracking cycle graphical environment might allow the attending physician to track the patient's recovery more fully and determine whether the planned restoration will be achieved.

The systems, methods, and devices disclosed herein provide various solutions to the above-described problems in the existing framework. The systems, methods, and devices disclosed herein optimize care and reduce production time for personalized services and devices to make them available for patients with emergent or low-volume unique conditions (e.g., trauma, oncologic surgery, and cardiac, or neurosurgical interventions). Measuring and tracking restoration and failure rates and combining those with other known failure risks (e.g., radiation, poor nutrition, smoking, body mass index, operative time, vasculopathies, and chemotherapies that inhibit wound healing) in a patient-specific manner could lead to improved Virtual Surgical Planning (VSP) and device design, and potentially, become “standard-of-care”. The knowledge that some failures, through tracking, could be anticipated and avoid secondary surgeries, inspires this study to see if the initial procedure could be reinforced by ICME, MM, and Hybrid Autonomous Manufacturing (HAM) to assist the manufacturing engineers and the attending physician on the optimal fixation design and any risks should be tracked in the event of weak or non-union. The planning, fabrication, and tracking process could all be accomplished at the point-of-care in real-time. Equally useful would-be research into local fabrication (i.e., distributed manufacturing) methods that make continuous improvements in these procedures as well as treatment outcomes.

### Challenges Facing Integration of Device and Fabrication Process Optimization for CMF Fixation Hardware

The cutting-edge practice to personalize off-the-shelf CMF reconstruction fixation plates to fit the patients' anatomy is by iterative manual bending of a plate by the attending surgeon to fit a 3D printed model derived from Virtual Surgical Planning (VSP). This operation is time-consuming, less effective, and lacks precision (repeatability), which can ultimately lead to sub-optimal plate shape and placement. Additionally, repeated bending might provoke metal hardening, fatigue, and reduction in the mechanical properties of the bent areas. To solve these problems, the attention of medical research has focused on Additive Manufacturing (AM), better known as 3D printing, for pre-operative treatment planning. This set of techniques is commonly associated with personalized components and increased freedom of design. Since the final form is generated, no bending is needed. However, the expense of fabrication remains significantly more expensive. Despite the expense, 3D printed plates are now the standard-of-care for many CMF surgeons whose clientele can reimburse the high cost, and most importantly, whose care can accommodate the 3 weeks to 3 months of current vendors. Some of the most interesting advantages of 3D printing over traditional manufacturing methods include the potential reduction in manufacturing times and the variety of materials that can be used. However, those cost reductions are not yet available and are not anticipated for undetermined years to come.

Future AM systems are likely to allow the design engineer to optimize mechanical properties. Also relevant is the Integrated Computational Materials Engineering (ICME) approach to link the underlying properties of the material with its intended performance, which could be translated to appropriate materials for the demands of the patient's local anatomy. With large CMF graft fixation, such capabilities would enable the modulation of personalized skeletal reconstruction hardware to avoid stress shielding and/or device stress concentration. This phenomenon occurs when there is an elastic mismatch between the bone and the adjacent metallic implant, causing the transfer of biomechanical load to the implant. The reconstructed tissue may heal but subsequently receives less stress than is needed to maintain the bone, leading to mass loss and possibly mechanical failure. Nevertheless, the surface roughness, mechanical strength, and chemical composition of the final 3D printed part may lead to a cytotoxic and inflammatory response, as well as anticipated failure within the body. Moreover, the adoption of 3D printing technology must comply with fabrication standards and regulatory agency oversight, such as the FDA. In addition, the limited availability and high cost of 3D metal printing limits its use at this time at most medical facilities.

It is currently considered state-of-the-art at tertiary and quaternary care medical centers, to 3D print models of the patient's reconstructed anatomy for use as a target substrate for pre-operative manual fixation hardware bending. At very few advanced quaternary referral centers, such as the Mayo Clinic, it is possible to perform 3D printed anatomic models for bending plates, 3D printed fixation trays for osseous reconstruction, and also perform real-time Virtual Surgical Planning (VSP) and directly 3D print personalized Ti-6Al-4V fixation plates for CMF reconstructive surgeries. However, as mentioned, manual bending can be imprecise (i.e., leading to gaps with the bone), make plan screw depth planning difficult, work harden crimp points, which may lead to future fatigue failure, and be time-consuming.

Considering the vast majority of CMF fixation hardware, which is prepared by manual bending, from the perspective of industrial metal forming, one would identify a lack of flexibility and modularity to generate personalized plates with both optimal shape and mechanical performance determined via VSP. For instance, current workflows utilizing standard manufacturing processes such as incremental sheet forming, multi-point roller-bending, and reconfigurable or rapid forming dies, allow a high degree of relatively low-cost modularity in current industrial production of customized metallic parts. The limitations of these processes derive from the need for generic tooling geometries and the associated manufacturing costs. However, this disclosure translates the advantages of these many forming processes to find a feasible workflow in the CMF fixation hardware manufacturing process that also results in optimally performing devices.

### Need for an Integrated Computational Materials Engineering Strategy for CMF Fixation Hardware

Reportedly, 36-39% of hemi-mandibular graft fixation devices can be expected to fail and require revision surgery, and 8-10% of all CMF fixation plates have been observed to break, loosen, or in other ways fail during normal activities. In addition to the painful emergency caused by the unexpected failure of these devices, typical re-operation costs an average of $50,000. This is an example of current procedures not having sufficient (1) mechanical input into the selection of off-the-shelf devices or materials, (2) no way for the attending surgeon to visualize the mechanical performance given the available choice of where to place the device, its material, or its shape, and to use their medical judgment to determine what is best for their patient, and (3) the simple limitation of manually not being able to bend a fixation plate sufficiently to achieve a flush fit to the underlying bone.

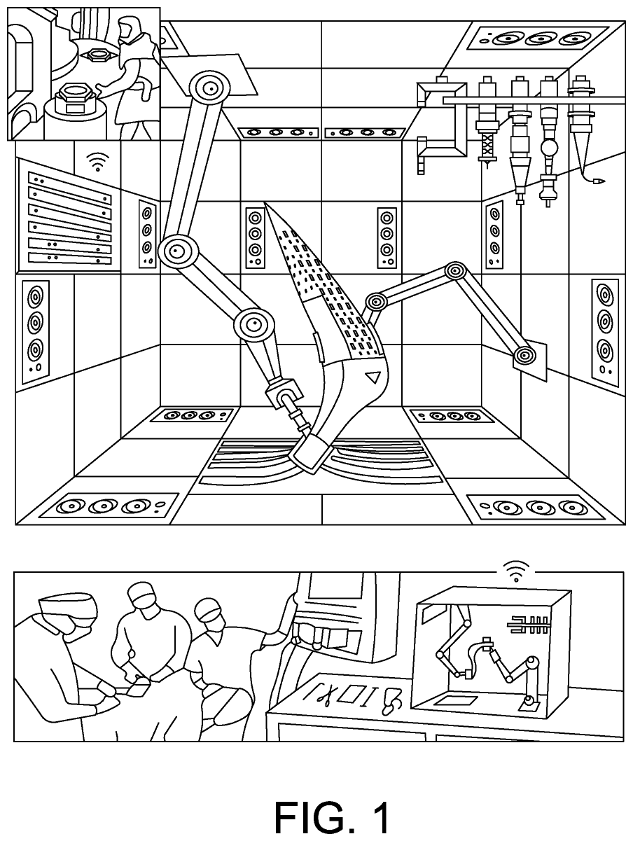

Existing options for personalized CMF reconstructive surgery by in large involve a combination of art (of medicine) and surgeon experience. Such work is based more on medical judgment than it is model-driven science. To go from the gold-standard manual plate bending based on prior experience with fixation devices and attempts at best-fit to an ICME approach that considers a model-based definition of design optimization, materials, and manufacturing processes, Point-of-Care Manufacturing (POCM) would benefit from an MM and Hybrid Autonomous Manufacturing (HAM) workflow that includes software-driven decision making to assist the surgeons with design and fabrication optimization. The latter would be guided via Virtual Surgical Planning (VSP) software and implemented by an MM-HAM system as an approach to bring together materials and processes with novel sensing and artificial intelligence. That conceptual approach could begin with the design, in a test case of mandibular graft fixation, of an optimal fixation plate that could be fabricated with flexible and automated metal forming processes. This latter part mimics what an experienced artisan or surgeon might do in the OR, but because the process would be automated it can better adhere to a device design derived from work in VSP software based on the surgeon's best judgment and rules of best practice and considering the biomechanical modeling of healing outcomes irrespective of surgical experience. In addition, by using MM to control and record the exact manufacturing sequence by means of sensors and robotic manipulation systems, the fabricated parts would better adhere to quality standards. For example, see FIG. 1 showing Hybrid Autonomous Manufacturing (HAM): Point-of-Care Manufacturing (POCM) of a hip implant via robotic machining with sensors (upper), and a schematic of POCM HAM robots near OR (lower).

The accuracy that can be achieved on existing robotic bending machines by integrating bending sensors to support angle correction, spring back compensation, or material thickness variation is an example that demonstrates the robustness and benefits of integrating sensor systems into metal forming processes. Other sensor systems available commercially include optical, infrared, and x-ray imaging devices to measure residual stress or texture, which could be incorporated into a feedback loop with an environmental control system to achieve the desired material properties and geometry. The key to these advances will be developing algorithms or procedures, likely enhanced with AI and ML, and controlling both the immediate process and the process sequence, for the automated fabrication of fixation plates, which may reduce manufacturing times, procedure cost, risk of re-operation, and near-term risk (e.g., by reducing OR time and procedure precision).

Thus, the motivation of the present disclosure is focused on presenting a hypothetical Point-of-Care Manufacturing (POCM) workflow that could improve the manufacture of CMF fixation hardware considering the approach of ICME and embracing HAM and MM. In addition to workflow development, the physical challenges encountered in the hybrid (i.e., multi-method) fabrication of fixation hardware will be discussed in a proof of concept. Finally, the challenges that the full realization of the workflow will take will be discussed. The provided examples will focus on mandibular graft fixation. However, the following could be quickly generalized to skeletal reconstruction devices of many kinds and the approach to other medical device POCM.

### Example System, Method, and Proposed Workflow

In order to propose a workflow that considers the approach of ICME in a Virtual Surgical Planning (VSP) environment, relevant aspects related to the requirements for the design of fixation plates were obtained from close consultation with stakeholders (e.g., surgeons, manufacturing engineers, and VSP software providers). One of them was selecting strategies focused on bone gap reduction to ensure that the plate is in close contact with the underlying bone to increase fixation stability. The tools envisioned for the workflow should also consider biomechanical data to choose the optimal fixation plate shape, thickness, length, footprint, and location, as well as fixation bone screw location, type, and length. All these variables play a biomechanical role in bone healing as well as the healed bone's subsequent ability to fully restore function.

The model for most commercially available services maintains confidentiality of the workflow used between obtaining a CT scan of the patient and then presenting the fixation hardware to the physician for approval. While the physician must approve, it is not usual for them to request comparative results based on varying location, material, shape of the fixation plate, or screw depth and location. Standards used by FDA panels for these devices include ASTM F382-17. This standard establishes consistent methods to classify and define the geometric and performance characteristics of bone fixation plates.

With this information, a study then proceeded to design a feasible workflow for the manufacturing of fixation plates. Here, the study devised a workflow that would facilitate the local manufacture of mandibular fixation plates starting with reliable metal forming techniques and a plan to move to more sophisticated equipment. The disclosed workflow considers the use of (a) VSP to guarantee an implant design that is flush with the bone surface and with optimal biomechanical performance during bone healing, (b) process engineering for a stepwise ICME approach, and (c) uniform deformation strategies to reduce localized work hardening (e.g., at thinned crimp points in current off-the-shelf devices) that risk subsequent fatigue failure of the fixation plate. The closed-loop fabrication (i.e., feedback between design, fabrication, and functional outcome) would be made possible by considering the above-mentioned approach of Hybrid Autonomous Manufacturing (HAM) and MM. Also, fabrication strategies that most efficiently reach the intended shape while maintaining desired mechanical properties would be considered.

### Hypothetical Framework for Robotic POCM of Mandibular Graft Fixation Plates

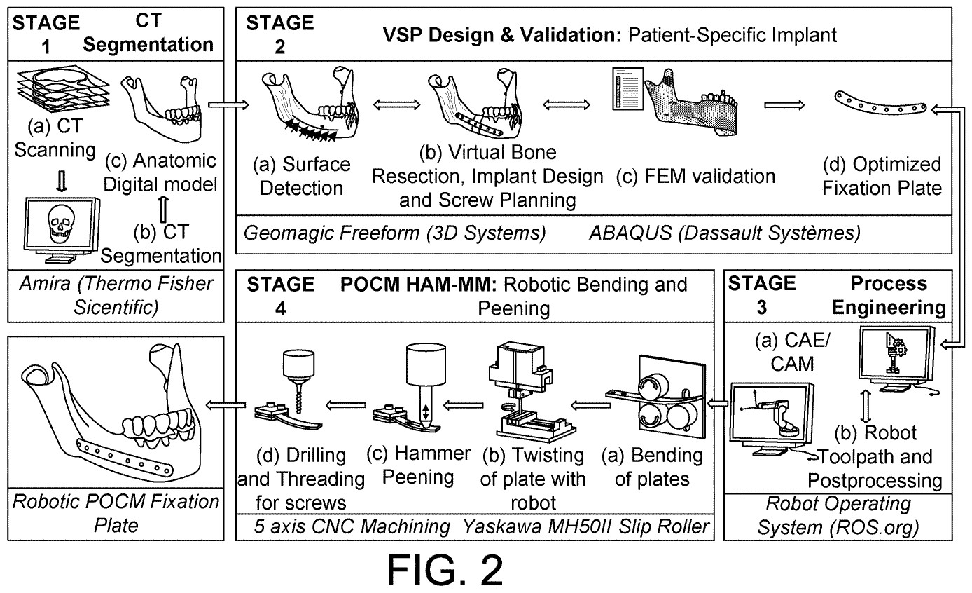

The disclosed hypothetical workflow includes four stages, as shown in FIG. 2.

Specifically, FIG. 2 shows a diagram of a hypothetical robotic Point-of-Care Manufacturing (POCM) workflow including Stage 1: Segmentation (identification) of patient 3D CT surfaces of interest; Stage 2: Implant Design and Mechanical Modeling: Validation of plate location, fit, and screw paths; Stage 3: Manufacturing Process Engineering coded robot tool paths; Stage 4: Bending, Twisting, and Peening leads to a flush-fitting fixation plate.

The process starts with the CT scan of the region of interest and the segmentation of the anatomical surfaces to be reconstructed. Then, the bone model is processed in a Virtual Surgical Planning (VSP) environment as in surgery (i.e., cut, reconstructed, engrafted, etc.). In this virtual environment, it would be possible to design a personalized implant to fit the original or reconstructed anatomy. Afterward, the mechanical performance of the reconstructed, and fixated, bone graft would be computationally assessed (i.e., applying a static load) via Finite Element Analysis (FEA) and further optimized to enhance the surgical outcome. While most relevant studies consider the design of patient-specific implants and cutting guides, herein this study includes the personalization of a medical device based on the mechanical requirements. Finally, the manufacturing process would be based on metal forming strategies, which would be previously validated via process engineering, to ensure personalized devices with desired mechanical properties as an outcome.

It is important to mention that in these stages the study considered the use of a quality management system (QMS) not only for the fixation plates that would be manufactured but also for the hardware and software considered in the workflow and the manufacturing process. In this sense, design and fabrication processes would be documented to maintain their effectiveness according to the requirements of international standards, such as ISO 13485:2016 (i.e., Medical Device Good Manufacturing Process) for medical devices.

### Stage 2 (Medical Device Shape and Mechanical Personalization)

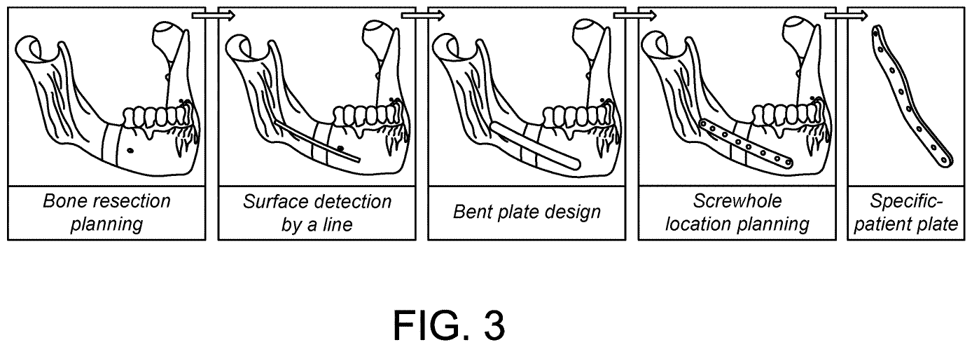

Patient-specific mandibular graft fixation may be designed in Geomagic Freeform (3D Systems, Rock Hill, SC, US) software. The software allows the detection of the mandibular surface. A generic straight fixation plate design may be used to create a bent plate that is fully in contact (flush) with the mandibular and graft surfaces. The Geomagic digital tools also facilitate a virtual surgery simulation that considers the location of the mandibular resection, geometry, and length of the bone graft during implant design. FIG. 3 shows a workflow for designing a specific-patient mandibular fixation plate. A line, which would be the undersurface centerline of the implant, is drawn over the bone surface (e.g., on the mandibular and bone-graft (i.e., osteotomy) surfaces which represents the midline of the desired fixation plate's location). Next, the implant design is warped based on that line. A series of operations would be used to create the personalized plate with the desired external dimensions (i.e. length, width, and thickness) based on the mechanical needs of the healing process as well as consideration towards not interrupting future normal loading of the healed bone. Then, the mechanical modeling includes planning screw and screw hole location and length. Finally, cutting guides would be designed. If useful, the host mandible, bone graft, screws, and implant could be exported for testing and optimization, by computational and/or in-vitro mechanical analysis, of the overall reconstruction's mechanical performance during mock chewing. The main purpose of the fixation plate is to hold in close contact the graft bone with the host mandible and to offer stability to the graft union.

The mechanical behavior and strength of the fixation plate, fixated bone graft, and host mandible would be simulated during mastication via static FEA for two scenarios of interest: (1) during the healing period to evaluate the implant's stiffness and stability, and (2) after bone healing and muscle force restoration is complete to avoid stress shielding of that newly healed bone. To this end, it would be necessary to create a volume mesh of the 3D CT or CAD-derived components (fixation plate, host bone, screws, and bone graft), set the boundary conditions (displacement restraints and forces), material properties, establish the interaction between components and solve the model. Furthermore, preliminary mesh quality and mesh convergence studies must be performed to increase the accuracy of the FEA results. Boundary conditions simulate chewing for maximum occlusion at the right first molar (M1) by restraining the movement in all directions of the buccal cusps of the teeth when they are inside the two rows of upper cusps (i.e., centric occlusion). The mandibular condyles would be constrained to prevent movement as well. Each masticatory muscle's force magnitude, direction, and area of attachment would be defined. However, 60% of the maximum value would be used during the before-healing computational analysis as chewing power decreases after mandibular reconstructive surgery and is slowly regained.

The contact between the host bone and the grafted bone would be simulated as well for two scenarios: before-healing (no union) and after-healing (union). Others have analyzed the pre-healing state of engrafted bone by assigning a friction coefficient of zero between components to allow free motion. After computational analysis, the host bone-graft bone interface micromotion and reaction force, as well as the resulting Von-Misses stress distribution in the bone and implant, would give feedback to the plate design stage for implant optimization. With these results, it could be assured that the bone-graft is in compression and the maximum micromotion value (300-400 um) is reached, both critical for successful healing. Additionally, the stress distribution results would show the location of stress concentrations and thereby potential areas of failure to optimize (remove) in the design of the skeletal fixation plate. Thus, the process of iterative design of the plate, screw depth, location planning, and validation by mechanical testing would ensure prior to implantation that the performance-optimized plate was obtained by the optimized POCM process.

This pre-operative mechanical model of chewing could be used to interactively change the size, shape, or location of the fixation device, which has been demonstrated to have an impact on the reduction of stress shielding in implants. These variations are all done to accomplish three things simultaneously: optimal healing outcome; post-healing lack of stress shielding; and fabrication process engineering designed to achieve both the personalized shape and mechanical function of the fixation plate. In the ideal situation, the patient's surgeon would have input into these decisions. That rarely occurs in current commercially available service workflows. When personalized plates are ordered from commercial vendors, the physician's input may be limited to approval of the device's final shape for delivery.

### Stage 3 (Process Engineering)

Once the optimized design of the fixation plate is obtained, manufacturing planning can proceed (e.g., process engineering, see FIG. 2). At this point, the curvature ranges and twisting angles of the plate would be determined from the optimized design and mechanical simulation performed in stage 2. To fabricate the fixation plates, the deformation strategy to obtain the primary shape would be performed by roll bending. Inspired by the performance of automated and flexible metal forming techniques, specific deformations or twisting may be performed on the plate with a robotic system to achieve the shape determined in the previous stage.

This back-and-forth stage would also serve to validate the optimization of the fixation plate's performance and optimization of the fabrication process to produce a fixation plate with that performance. This would be accomplished by using an ICME validation model to help predict the microstructural evolution of the plate's material based on the design variables and forming process parameters. This data would also help determine, through computational simulations of the metal forming process, the forming loads, spring back, or specialized fixturing for the available plate bending equipment. The simulation and validation of robot trajectories and forces for the fabrication of fixation plates would be translated into Robot Operating System (ROS) process controls. This would allow a digital twin to validate the manufacturing of the plates obtained from the design stage.

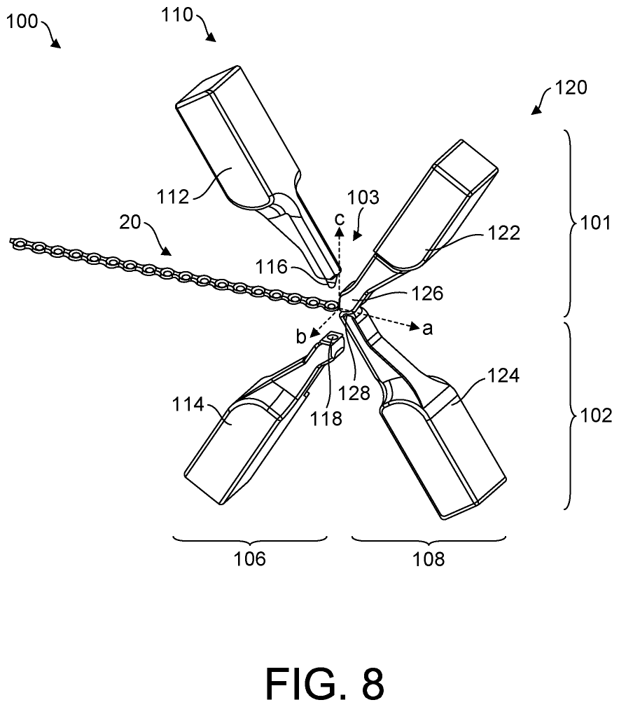

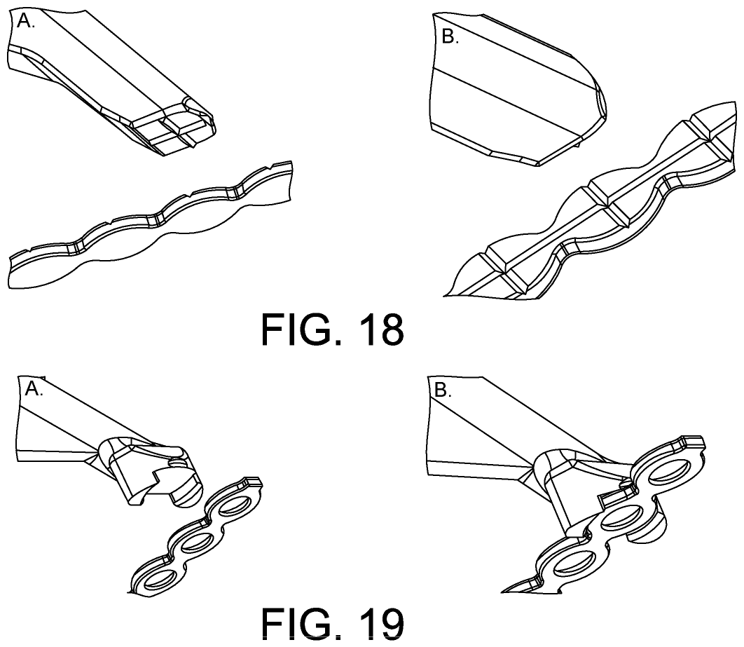

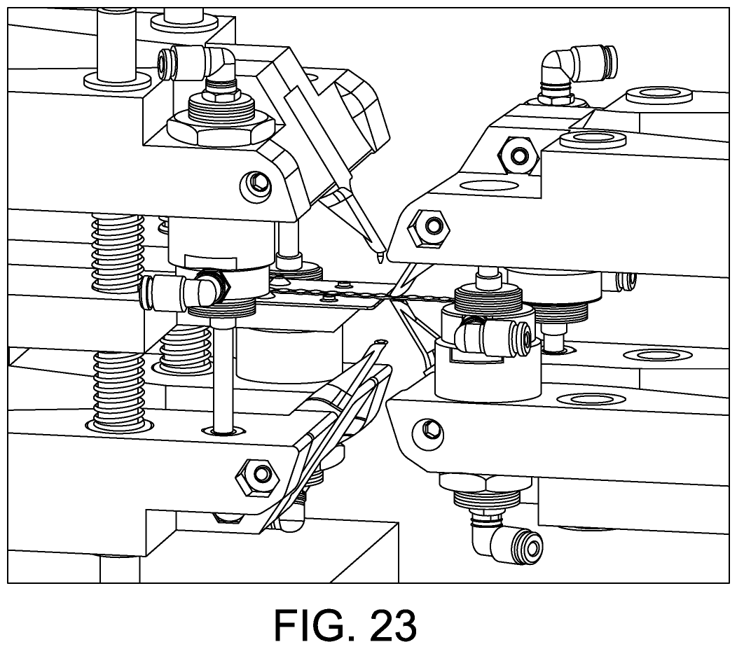

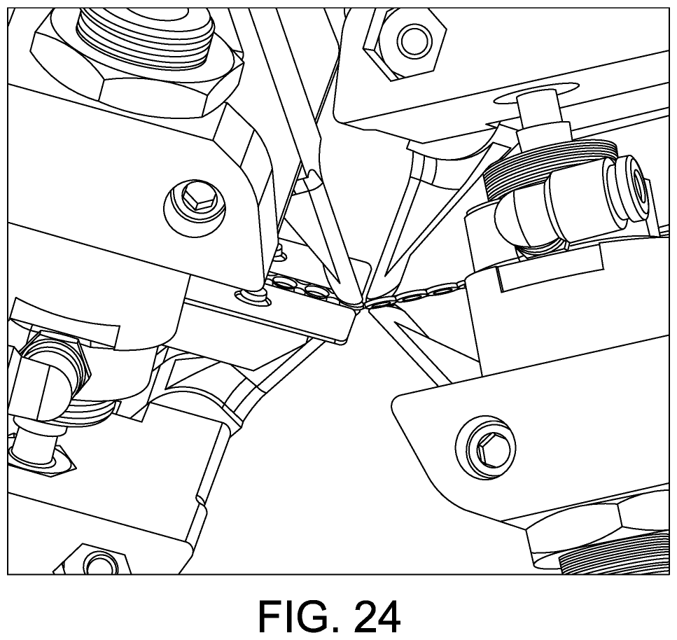

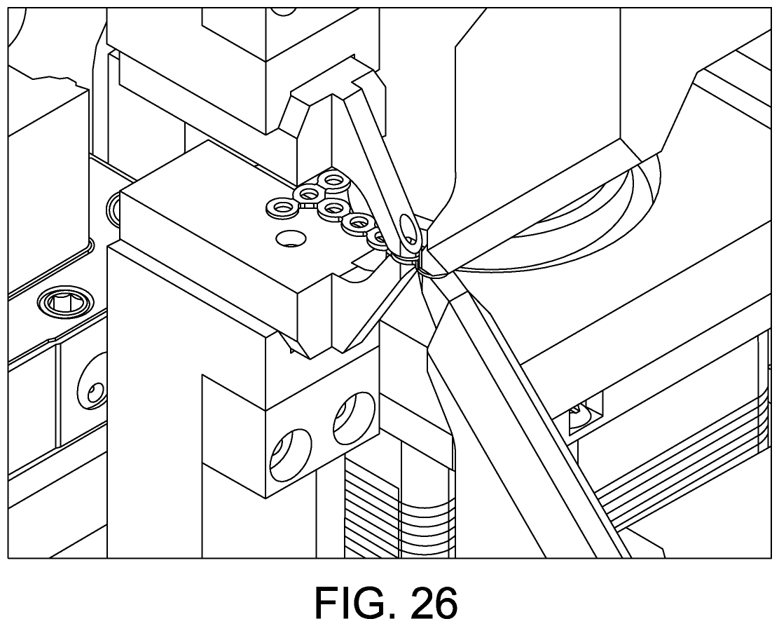

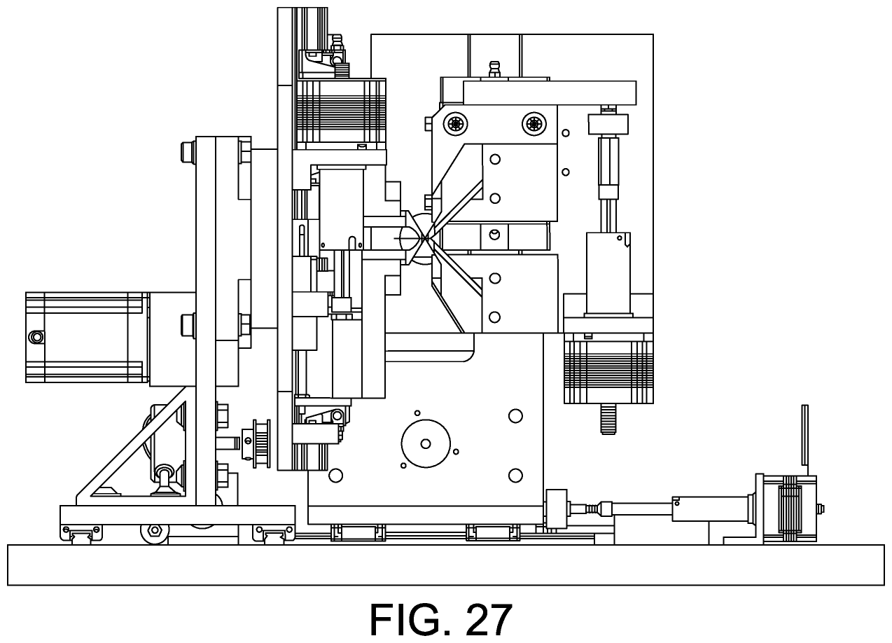

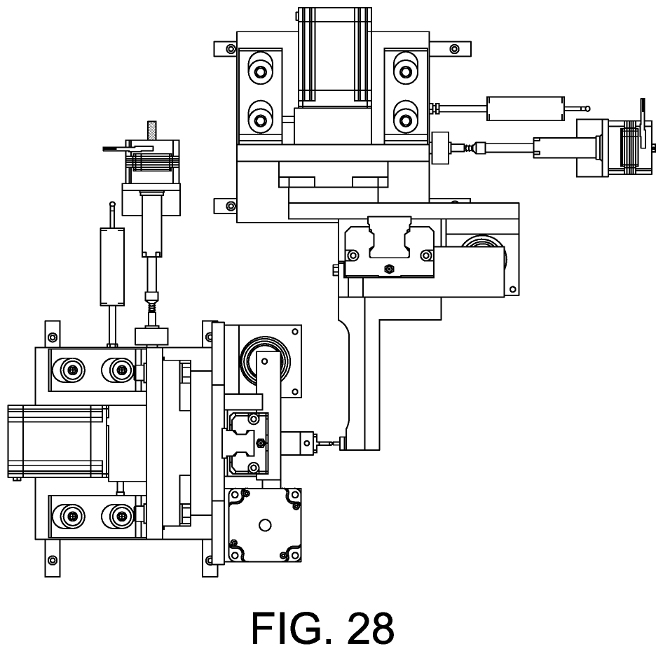

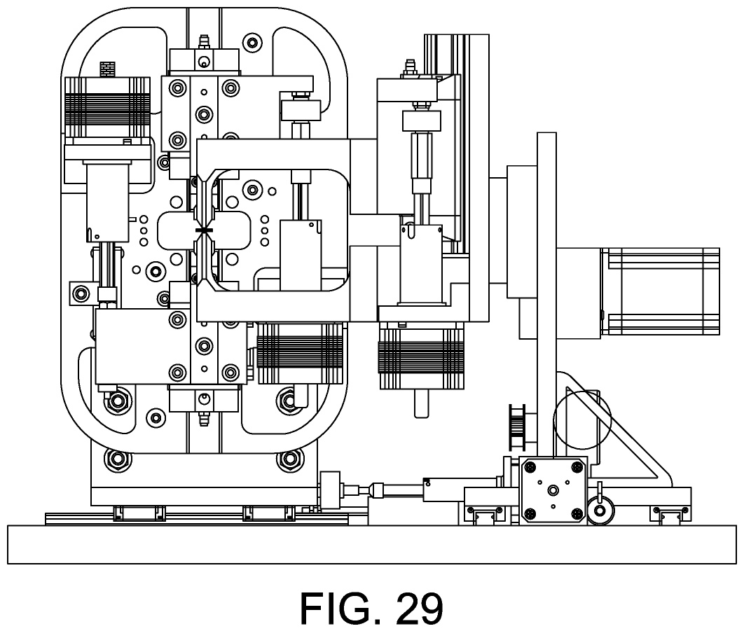

One example of a Stage 3 device, method, and system is shown in the below-described Example #2, shown in FIGS. 8-17, with additional examples shown in FIGS. 18-30. This example bending device and system provides one example of bending a medical implant (e.g., a fixation plate) to a desired angle and topology. The systems and devices of FIGS. 8-30 can accept and read instructions based on the above-described steps (e.g., Stage 1 and Stage 2 data) and perform operations to shape the fixation plate to match a patient's anatomy. The systems and devices of FIGS. 8-30 may further implement additional features and structures elsewhere described herein (e.g., after-treatment operations, metal hardening, and/or other microstructure-related operations).

### Stage 4 (POCM: Fabrication Modalities and Sequencing for Deformation of Graft Fixation Plate)

This stage consists of 4 sequential strategies based on a HAM-MM approach, that would produce the final fixation plate to design specification and considering the forming loads determined in the previous stage. The first step would consider bending straight plates by employing a slip roller of varying diameters according to the curvature ranges determined in the design phase. Then the plate would be delivered to a station and fixed in a vise press, so the robotic system can apply the determined loads and angles to twist it. The final rough tuning of the surface contacting the bone (to adapt the surface of the plate to that of the mandible) would be made by peening. This operation should minimize the space between the two surfaces for proper fixation. The eyelet for fixation screws would be threaded (i.e., either standard threading or locking head threading) using 5-axis CNC machining. The location of each hole is determined by the previous Virtual Surgical Planning (VSP). In addition, air gas will be used for chip removal during the process to prevent the cutting tool or plate from cracking due to material entrapment. Finally, the ends of the plate would be cut and polished to achieve the final geometry. It is important to mention that sensory systems and control algorithms (MM approach) would be used to track the fabrication process to ensure that the plates better adhere to quality standards and performance requirements identified in the VSP stage.

### Discussion

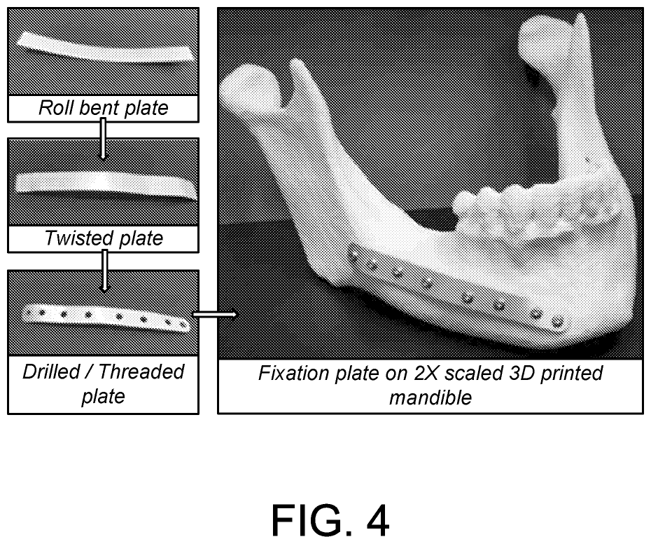

The hypothetical workflow envisioned in this section of this disclosure implies significant challenges for designing and fabricating personalized CMF fixation plates at the point-of-care. Thanks to a brief series of experiments where a study used a 3D printed 2× scaled model of a jaw to exemplify the fitting of a fixation plate, the study has begun to address some of the challenges that the real-world application of the proposed workflow would have to overcome. FIG. 4 shows the stages of the plate fabrication process. Specifically, FIG. 4 shows a step sequence for the manufacture of the aluminum fixation plate. First, through roll bending, the deformation is done to match preliminary the shape of the mandible. Second, the robotic twisting is based on the angles determined in the design stage. Finally, peening, drilling, and threading will be done on the plate for bone fixation. For one demonstration case, the study used a highly malleable strip of aluminum.

Starting with Virtual Surgical Planning (VSP) stage 2 for fixation plate design and fabrication planning, some of the challenges identified would be related to the iterative nature of the design and simulation software. For clinical cases with sensitive timelines, implant optimization could take considerable time. This stage also depends on the complexity of the case being handled. In addition, the computational processing required in FEA operations demands high-resolution meshes for both shape and mechanical optimization of the fixation plate. Such challenges must be considered to ensure the optimized design of a plate in a short time.

In addition to the proposed workflow (FIG. 3), obstacles exist in implementing a planned fabrication procedure that would prevent the removal of undesirable gaps between the fixation plate and the underlying bone. See FIG. 5, showing gaps between the fixation plate and host bone found in the designed and the proof-of-concept manufactured plate. For example, one obstacle might be the quantitative assessment of the required torque to deform the plates at different angles while predicting springback. This is especially relevant when working with materials with high stiffness, such as medical titanium or stainless-steel alloys. To achieve this, future work would address the development of an ICME model validation to predict microstructural changes during the forming process that might affect the performance of the plate, as well as computational simulation that offers a realistic environment of the manufacturing processes in the disclosed Point-of-Care Manufacturing (POCM) workflow.

Furthermore, real-time tracking of shape evolution and mechanical properties during machining, forging, and bending will be incorporated with a camera and tactile systems as an MM approach. Indeed, kinematic sensors in the robotic manipulation arms and a relatively few points that are tracked on the evolving fixation plate should be sufficient. In this way, the desired final mechanical properties can be conserved during fabrication. Experiments are also underway to ensure a flush fit of the fixation plate to the underlying bone.

These processes will require further experimentation as the kinematic adjustment of deformation and torque control strategies for 3D contours might become too complex to initially implement during metal deformation. Even though the goal of this effort is to manufacture, handle, and deform surgical-grade titanium alloy (Ti-6A1-4V) fixation plates, the study used relatively plastic (easily deformable), large-format, aluminum plates to test the design and manufacturing workflow. Titanium (Ti) alloys show higher hardness, stiffness, and strength than Al alloys. Furthermore, the deformation of Ti by a forming process can induce the development of texture deformation and changes in its mechanical properties. However, related work on cold roll bending of Ti has shown that it does not reduce the ultimate tensile stress or the hardness of the material, on the contrary, it would increase it. Other work has computationally demonstrated the alteration of the stress-strain path, hence the reduction of stress-shielding, by changing the material, location, or geometry of the implant. To get a better understanding of the resulting properties, forces, and torque that will be required for plate shaping and performance, computation and experimental studies that offer data for accurate deformation modeling (particularly in the process engineering stage 3) will be needed and demonstrated in a future study. This strategy would allow the determination in advance of the required changes to the manufacturing or fixturing process at stage 4.

The proposed workflow and other examples of this disclosure relate to and include a project titled Hybrid Autonomous Manufacturing Moving from Evolution to Revolution (HAMMER). A long-term goal of the HAMMER project is to develop general rules for product and process design and execution and to extract the salient features so that they can be used with varied equipment suites to instantiate workflows such as the mandibular graft fixation design and fabrication presented in this disclosure. This decision-making and learning process is the essence of what human artisans do, and if this can be accomplished digitally for instance by robots, it will be an approach of reinforcement learning. The designing of personalized medical devices and fabrication with a suite of computer-controlled machines would be complex but may be aided using ML and AI in stages 2-4 in the disclosed workflow. For complex open design problems that may have multiple valid solutions, appropriate AI and ML algorithms can quickly arrive at a valid fabrication solution that fulfills all design requirements. As algorithms are presented with more data for learning, they may produce designs faster, require fewer computational resources, and create more generalized designs, not rigidly constrained to a single use case. Understanding generalized designs may allow the future creation of billets that work well in POCM systems. The use of limited design procedures for well-documented billets will allow the creation of an FDA-approvable design envelope that can be tied to well-defined, quantitative medical indications.

After the optimized design process is complete, AI and ML could also be utilized to create a manufacturing schedule and coordinate machines to produce the required design quickly and efficiently. When leveraging complex machines with several degrees of freedom, AI and ML can calculate forward kinematics and move a workpiece from initial stock, through each manufacturing process, to the final geometry. Investing in the integration of AI and ML into POCM may improve the efficiency and efficacy of manufacturing personalized medical devices.

Authentication of raw materials and quality assurance of the final parts will also be a challenge for the widespread adoption of Point-of-Care Manufacturing (POCM). Each step will need to be accomplished under professional verification and validation (e.g., ISO 13485). For many products, quality assurance is performed on stock material, and the manufacturing method is certified. By measuring the quality of stock and certifying the process, it is possible to predict quality and deliver components that meet safety and performance standards. While producing high-mix low-volume components, the high variability of POCM processes used to produce each unique component will be incorporated into an FDA-approved Quality Management System.

### Conclusion

The current standard-of-care practice of manual bending an off-the-shelf skeletal fixation to fit a patient's anatomy in the OR, or a VSP-generated model usually requires multiple bending steps that are concentrated at given locations in commonly available fixation plates. The ability to smoothly curve a plate and gain a flush fit can be extremely challenging. Fatigue resistance may be compromised by excessive loading (i.e., stress concentrations) at gaps between the implant and bone, tensile residual stress at the maximum tensile region in the implant, and stress concentrations caused by kinks in bending. These factors increase the risk of failure for fixation devices on patients.

All of these problems may be minimized by the integration of an engineering approach to design, mechanical optimization based on expected performance, and an automated fabrication process. It also would be useful to provide surgeons and engineers with an FDA-approved design and manufacturing environment that allows them to optimize device location, material, and shape. Thus, this disclosure presents a hypothetical POCM workflow that improves the manufacturing of CMF fixation hardware considering the approach of ICME, MM, and englobing Hybrid Autonomous Manufacturing (HAM). This approach also leverages MM to design a personalized device's shape simultaneously with its function and a fabrication strategy using manufacturing modalities and device materials that can insure the output of a device with optimal shape and mechanical performance.

The proposed workflow, which consists of CT segmentation, Virtual Surgical Planning (VSP) design and validation, process engineering, and Point-of-Care Manufacturing (POCM) fabrication, would consider biomechanical data to choose the optimal fixation plate shape, thickness, length, footprint, and location, as well as fixation bone screw location, type, and length. All these variables play a biomechanical role in bone healing as well as the healed bone's subsequent ability to fully restore function and maintain itself long-term. This disclosure describes a process that begins with an interactive VSP derived from pre-operative patient 3D CT imaged surfaces. That environment would allow in the future a comparison of various mechanical outcomes driven by FEA and the physician input on: (1) fixation plate shape, (2) materials, and (3) performance. Once optimized, a fabrication process involving multiple (hybrid) metal forming methods could leverage technology that is significantly less expensive and more easily distributed than current 3D metal printing technology. This new data-driven POCM workflow would provide physicians and engineers confidence that they were optimizing therapeutic outcomes and beginning to close the loop between VSP and patient outcomes.

Although the proposed workflow is focused on hardware for mandibular graft fixation, it could be quickly generalized to skeletal reconstruction devices of many types and the approach to other medical device POCMs. Personalized (shape and location) POCM of fixation plates would benefit patients with rapidly emerging conditions such as skeletal reconstruction due to tumor or trauma who otherwise are unlikely to receive a personalized fixation device at most medical centers due to the need to surgically intervene in real-time.

### Example Device and System for Implant Formation and Bending

Disclosed herein is a device for forming and bending a medical implant (e.g., a bone fixation device), according to various implementations. The disclosed process and device geometry may be used in bending bone fixation plates by an automated process. The disclosed device may be implemented into the above-described workflow (e.g., in Stage 4 of the workflow in FIG. 2). For example, the disclosed device may accept a blank or unaltered bone fixation plate along with manufacturing instructions to bend the bone fixation plate to a desired topology. The desired topology may align with a model developed based on a patient's anatomy or a model of a specific medical device.

The disclosed device forms the desired medical device with accuracy and precision, performing each individual bending step a single time. Therefore, corrections to the bend angle (e.g., bend-back operations) are eliminated, reducing the fatigue created within the device. For example, the device is capable of bending each portion or section of a bone fixation plate with only a single bending operation. In some implementations, the disclosed device includes additional material hardening steps (e.g., peening or heat addition) or post-processing steps (e.g., drill or tapping holes). Overall, the disclosed device allows for an accurate and rapid bending operation for medical devices (e.g., medical fixation plates for reconstruction of damaged bones in a patient).

### Example Bone Fixation Plate

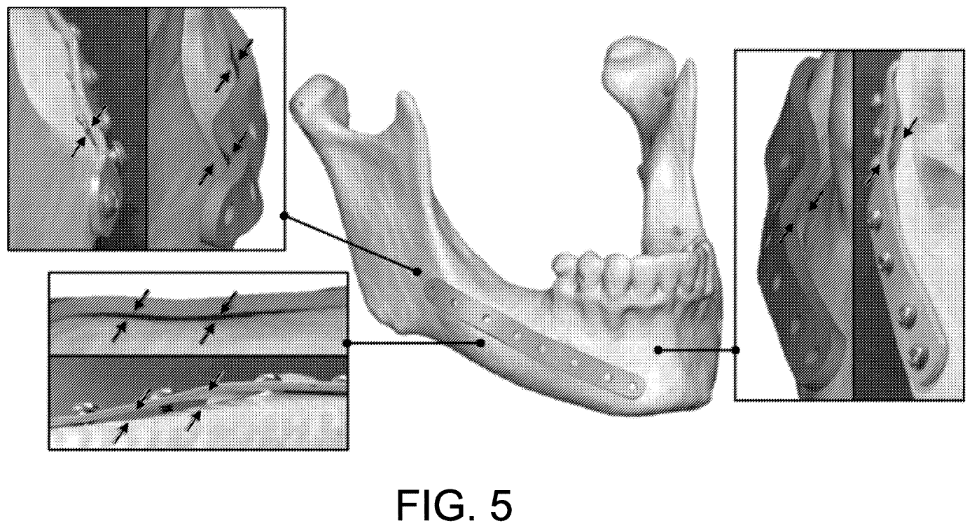

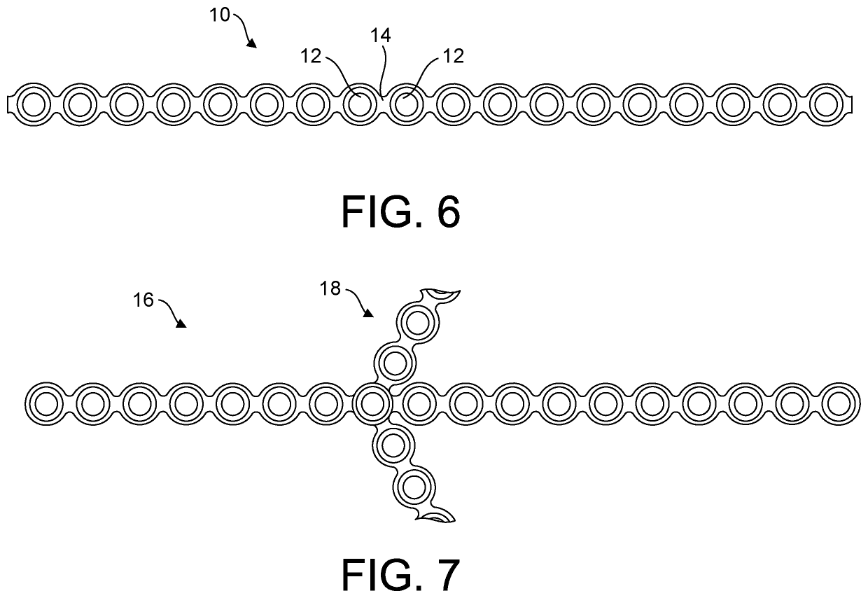

Surgical fixation plates ('fixation plates') are used to secure fractured bones to one another for proper healing. Fixation plates are typically made of a metal alloy that is bio-compatible (titanium and stainless-steel alloys are common). FIG. 6 provides the typical shape of a fixation plate 10, according to one example. The fixation plate 10 includes a linear sequence of eyelets 12 (nonlinear and branching versions are also available). These eyelets 12 permit passage of bone screws which join the fixation plate 10 to the bone surface. Fixation plates are typically supplied in a flat linear morphology, as shown in FIG. 6. Bone surfaces are composed of many complex curves and present few regions where a flat and straight fixation plate could be placed. In some procedures, the fixation plate 10 is joined to the bone via bone screws such that the axis of the eyelets 12 is normal to the surface of the bone. In such procedures, the fixation plates 10 are bent to match the curving surfaces of the bones to which they are joined. However, in other procedures and other implementations of the present disclosure, a bone fixation plate may have portions that do not match the curving surfaces of the bone or wherein the eyelets are not exactly normal to the bone surface (e.g., fixation plates extending across a void or defect; fixation plates coupled to rods or other implants). To facilitate bending, fixation plates typically include necked regions 14 between the eyelets 12 where bends and twists can be applied without distortion of the eyelets 12.

Bending of the fixation plates is typically performed by the attending surgeon while the patient is open on the operating table. The plate is manually bent and checked against the patient's bone in an iterative process until an adequate fit is achieved. This process is time-consuming, time which adds stress to the patient's body and drives up the cost of the operation. Larger bones require thicker fixation plates, such thick plates can be difficult for a surgeon to bend to proper shape, possibly resulting in tired hands. Additionally, repeated bending of the plate can induce damage, such as cracks, kinks, or surface defects. Excessive work hardening between any pair of eyelets will reduce the capacity of the fixation plate to bear repeated stresses, possibly leading to fracturing of the fixation plate by fatigue cracking in the months following surgery. Excessive work hardening of the fixation plate will occur anywhere where the surgeon has repeatedly bent the fixation plate in the process of finding a good fit. Unfortunately, a surgeon cannot know if any regions of the fixation plate have experienced excessive work hardening, leading to the implantation of plates with an unknown likelihood of failure.

For reconstruction of shattered bones in the patient's face an additional complication is introduced by the need to restore a patient's original appearance. The subtle bending of fixation plates to hold a patient's facial bones in their original positions takes additional time and skill on the part of the surgeon. 3D models of the patient's bones can be collected by CT or other medical scans. Those 3D models of the patient's damaged bones can be reconstructed in a computer where time and cost are less significant factors. The reconstructed facial bones of the patient can then be 3D printed and fixation plates can be bent to match prior to surgery. This technique is useful, however, there is a time delay for 3D printing. This time delay can be large, especially for 3D printed models manufactured and delivered from off-site. Because of these time delays, surgeries often proceed with stock fixation plates which will be bent during surgery.

In the operating room surgeons use specially designed plyers to grip and apply deformation (bending and twisting) to initially flat fixation plates. In principle, the general shape of any bone surface (or other surface) can be approximated by the application of bends and twists between the eyelets of a fixation plate. This principle extends beyond simple linear fixation plates of the type shown in FIG. 6. Branching and nonlinear (but still flat to the page) fixation plates (e.g., fixation plate 16 having branch 18 shown in FIG. 7) may be formed to match bone by this method. If the required angles for bending and twisting to match a patient's bone are known for each eyelet pair in a fixation plate, then it is possible to manufacture such a plate by an automated machine that can apply bends and twists between successive eyelets in an initially flat fixation plate. Such a machine would be capable of bending fixation plates to match computerized 3D reconstructions of patient bones without the need to wait for 3D prints and without the risk of surgeons overbending the plates. Such a machine would also take up the bulk of the physical effort, relieving the surgeon. For quality control purposes such a machine would be able to record and replicate the precise bends applied to any patient's fixation plate(s). This disclosure presents such a machine.

To produce an accurately bent fixation plate via an automated machine, the disclosed machine is able to grip adjacent eyelets on the fixation plate. In one implementation, the machine is equipped with opposing jaws having a geometry that allows for accurate indexing and constraining of the fixation plate while also allowing for bending without conflict between the jaws. The jaws have geometry that allows for gripping and bending anywhere along a fully or partially bent fixation plate without conflict between the plate geometry and the jaws. The jaw geometry is such that conflicts do not arise during the opening and closing of the jaws when bending an unbent, partially bent, or fully bent plate (i.e., the jaws or components thereof do not interfere with each other during a bending operation). At least one set of jaws may be opened at any state of bending for the purpose of indexing between eyelets.

### Example Bending Device

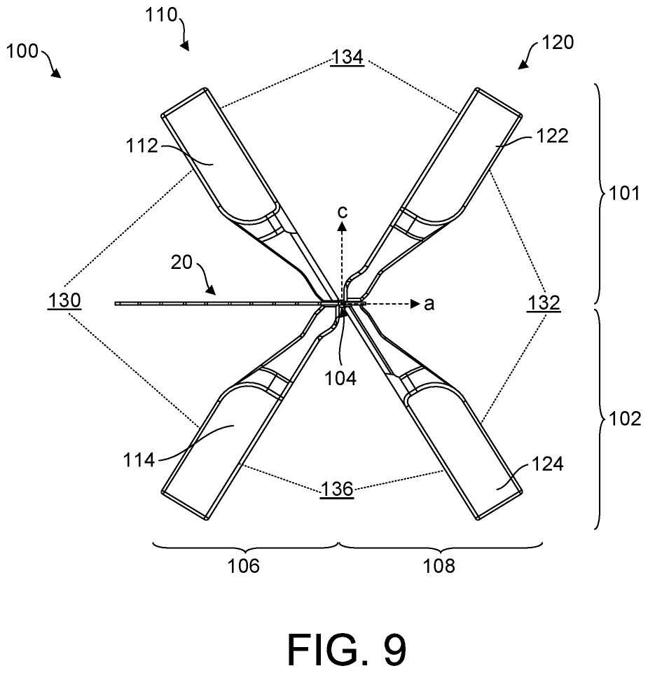

FIG. 8 shows a device 100 for bending a medical device (e.g., the bone fixation plates of FIG. 6 and FIG. 7), according to one implementation. As shown, the device 100 is engaged with a bone fixation plate 20 that is similar to the linear fixation plate of FIG. 6. The device 100 includes a first pair of opposing jaws 110 and a second pair of opposing jaws 120. The first pair of jaws 110 and the second pair of jaws 120 are generally arranged around a bending area 103 having a central bending point 104 for the device 100. The center of bending or twisting of the bone fixation plate 20 and the associated device 100 will always be between the two pairs of jaws 110, 120 at the central bending point 104 (i.e., a midpoint between the eyelets occupied by the two opposing indexing jaws). A side view of the device 100 is shown in FIG. 9 wherein each pair of jaws 110, 120 are engaged with an eyelet of the bone fixation plate 20.

The first pair of jaws 110 is disposed on a first end 106 of the device 100 (e.g., the input side of the device). The first pair of jaws 110 includes a first member 112 disposed on a first side 101 (e.g., an “upper” or “top” side) of the bending area 103 of the device 100. The first pair of jaws 110 further includes a second member 114 disposed on a second side 102 (e.g., a “lower” or “bottom” side) of the bending area 103 of the device. The first member 112 and the second member 114 extend away from the bending area 103 at an angle such that a first clearance area 130 is defined between the first member 112 and the second member 114. The first member 112 includes a first tip 116, and the second member 114 includes a second tip 118. The first tip 116 of the first member 112 is configured to engage with the second tip 118 of the second member 114. For example, the first tip 116 shown in FIG. 8 includes a protrusion, and the second tip 118 shown in FIG. 8 includes a corresponding cavity such that the first and second tips interlock with each other in the engaged configuration (e.g., a tooth-like engagement). However, in other implementations of the disclosed device, the first and second members of the first pair of jaws generally engage with each other on opposing sides of a medical device. For example, in some implementations, the first tip and the second tip engage through the eyelet, around the outside of the eyelet, or both. In other implementations, the first and second tip each provide a force on opposing sides of the medical device (e.g., bone fixation plate with or without an eyelet as in FIG. 18). In some implementations, the first and second tip engage via opposing forces without direct contact with each other.

The first member 112 may be an “indexing jaw” having a protrusion or “indexing tooth” used to locate the first pair of jaws 110 accurately in an eyelet of the fixation plate 20. The second member 114 may be a “clamping jaw” having a cavity or hole to accept a portion of the indexing tooth of the first member 112. The clamping jaw of the second member 114 may have a larger thickness than the indexing jaw of the first member 112 because of the hole. However, in other implementations, the first and second members engage in a different manner (e.g., via a smaller protrusion or a clamping structure around the bone fixation plate).

The device 100 may include an actuator configured to move the first pair of jaws 110 and/or the second pair of jaws 120 between (i) an engaged configuration wherein the first tip 116 engages the second tip 118 (e.g., as shown in FIG. 9), and (ii) a separated configuration wherein the first tip 116 is separated from the second tip 118 by a distance (e.g., as shown in FIG. 8). The actuator is also configured to rotate the second pair of jaws 120 to an angled configuration with respect to the bending area 103.

The first pair of jaws 110 is shown on the first end 106 of the device 100, which may be a “stock side” of the device 100. The stock side is named because of the stock version (or unbent version) of the bone fixation plate 20 entering the bending area 103. In contrast, the second pair of jaws 120 is shown on the second side 108 of the device 100, which may be a “product side” of the device 100. The product side is named because of the product, or final, version of the bone fixation plate 20 leaving the bending area 103.

The second pair of jaws 120 includes a third member 122 disposed on the first side 101 of the bending area 103 of the device 100. The second pair of jaws 120 furth includes a fourth member 124 disposed on the second side 102 of the bending area 103 of the device 100. The third member 122 and the fourth member 124 extend away from the bending area 103 at an angle such that a second clearance area 132 is defined between the third member 122 and the fourth member 124. The third member 122 includes a third tip 126, and the fourth member 124 includes a fourth tip 128. The third tip 126 of the third member 122 is configured to engage with the fourth tip 128 of the fourth member 124 (e.g., similar to the engagement described in the first pair of jaws 110).

The device 100 is configured to bend the bone fixation plate 20 to a desired angle and/or topology. For example, in the configuration shown in FIG. 9, the second pair of jaws 120 are movable to bend the first eyelet of the bone fixation plate 20 with respect to a second eyelet of the bone fixation plate 20 closer to the first side 101 of the device 100.

Furthermore, the angle at which the first member 112 and the third member 122 extend away from the bending area 103 further defines a third clearance area 134 defined between the first member 112 and the third member 122. Similarly, the angle at which the second member 114 an the fourth member 124 extend away from the bending area 103 further defines a fourth clearance area 136 defined between the second member 114 and the fourth member 124.

Generally, each of the clearance areas 130, 132, 134, and 136 provides a space for the members of the pair of jaws 110, 120 to bend with respect to each other without interference or contact. In some examples, the angle of one member from a vertical axis passing through the central bending point is between 10 and 80 degrees (e.g., 30 degrees). In other implementations, various angles of extension of the members are contemplated by this disclosure.

In use for a bending operation, the second pair of jaws 120 is movable to both translate and rotate as needed. For example, the second pair of jaws 120 may be rotated to an angled configuration about either axis shown in FIG. 8—axis “a”, axis “b”, or axis “c”. The second pair of jaws 120 may be rotated about the a-axis (e.g., a roll operation), the b-axis (e.g., a pitch operation), and/or the c-axis (e.g., a yaw operation). Rotation about multiple axes at once (either the same or varied angles/amplitude of rotation per axis) is contemplated by this disclosure. The central bending point 104 is at the origin of the axes.

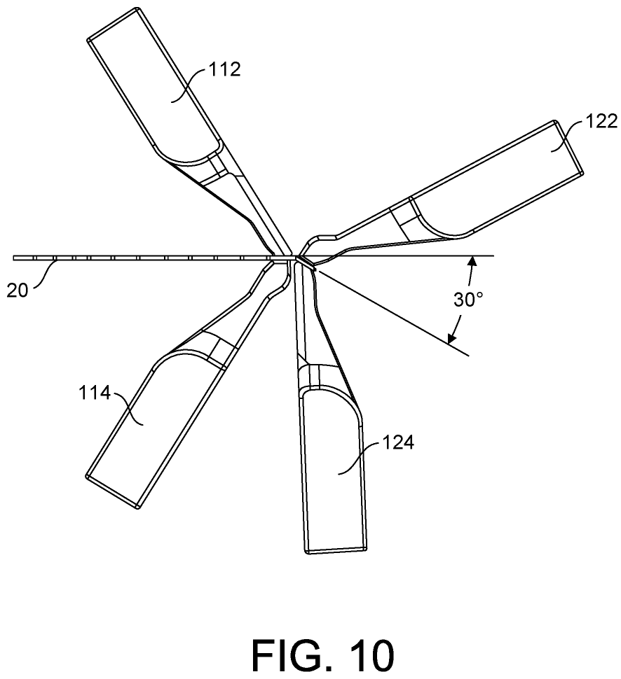

FIG. 10 shows the device 100 wherein the second pair of jaws 120 have been rotated about the b-axis (e.g., a pitch rotation or an out-of-plane bending operation). The maximum bend angle is limited by the front surfaces of the first and second pair of jaws 110, 120. For the geometry shown in FIG. 10, the maximum bend limit without jaw collisions is 30°. However, other angles are contemplated by this disclosure, and 30 degrees is illustrative only. Bends are placed by applying normal pressure to the fixation plate 20 from all four members 112, 114, 122, 124 of the first and second pair of jaws 110, 120. Then, the second pair of jaws 120 are rotated (e.g., by an actuator with an optional frame device coupled to the second pair of jaws 120) about the center of rotation/bending 103. This applies a highly localized deformation to the fixation plate 20 without distorting the plate's eyelets.

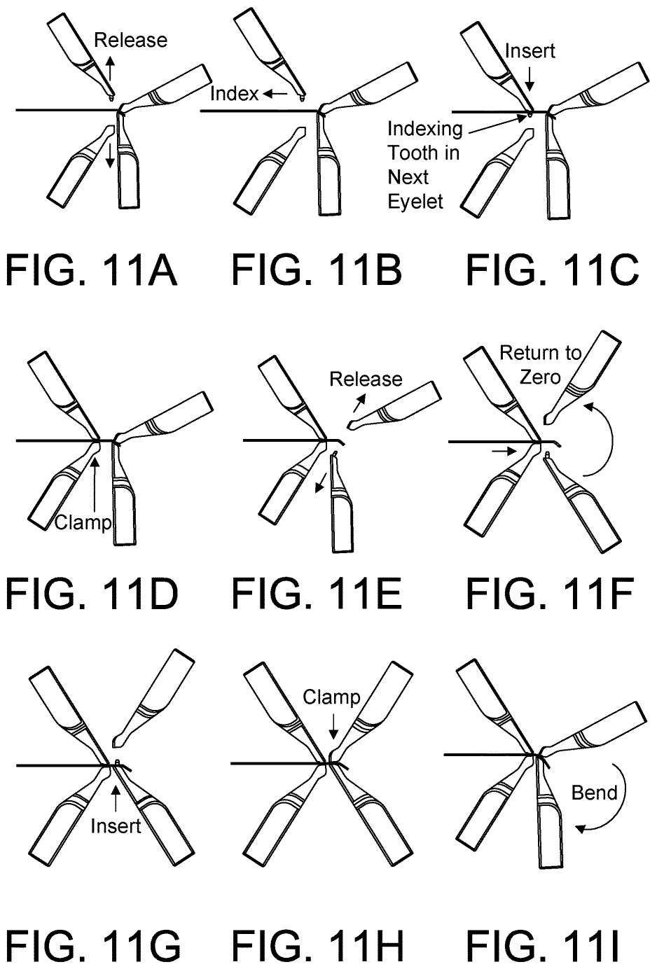

FIGS. 11A-11I show a sequence of steps for performing a second bending operation and subsequent bending operations (e.g., after the bending operation of FIG. 10). In FIG. 11A the first pair of jaws 110 (left) are shown in the open position; this state follows the bend placed in FIG. 10. Notice that in FIG. 11A the second member 114 and the fourth member 124 do not conflict during opening. In FIG. 11B the first pair of jaws 110 are translated to the left by the center-to-center distance of the plate's eyelets, leaving the first tip 116 (e.g., an indexing tooth) of the first pair of jaws 110 directly over an eyelet. In FIG. 11C, the first member 112 of the first pair of jaws 110 is moved down into contact with the fixation plate 20, and its indexing tooth is inserted into the eyelet. In FIG. 11D, clamping pressure is applied by the second member 114 of the first pair of jaws 110. In FIGS. 11E and 11F, the second pair of jaws 120 (right) are released and rotated back to the origin. In FIG. 11F, the bone fixation plate 20 is translated to the right by the first pair of jaws 110 by the distance between one eyelet pair. In FIGS. 11G, 11H, and 11I, the second pair of jaws 120 are indexed into, clamped onto, and then apply a second out-of-plane bend to the bone fixation plate 20. Additional steps and alternative bending angles, distances, and operations are further contemplated in other implementations of the device 100.

By the disclosed process, a sequence of clockwise or counterclockwise bends may be applied to a medical implant (e.g., the bone fixation plate between successive eyelet pairs).

Because the plate is always held rigidly by the first or second pair of jaws (or both), the fixation plate will always have a known position determined by at least one indexing tooth (or other indexing feature on the jaw tip). By the sequence of steps shown in FIGS. 11A-11I, a fixation plate may be ‘walked’ from the first end 106 (or stock side) to the second side 108 (or product side) of the device 100 where each eyelet pair receives a specified bend, resulting in a ready-to-be-implanted bone fixation plate.

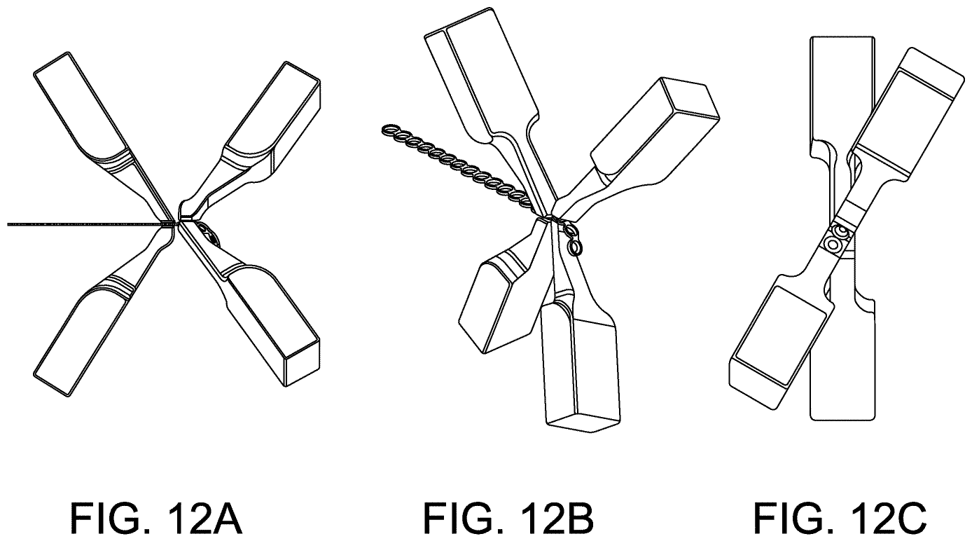

FIGS. 12A-12C show the device 100 with the second pair of jaws 120 performing a rotation about the a-axis (e.g., a roll operation). The second pair of jaws 120 are shown applying a 30° twist to the fixation plate 20 as one example of a twist angle. The mechanical limits on twisting are determined by the properties of the plate material, unlike with bending where the limits are a consequence of the jaw geometry. The process for applying twists between successive eyelets is similar to the process depicted in FIGS. 11A-11I for successive bends, where the equivalent step for FIG. 11F consists of jaw rotation back to the original position along a different rotation axis, that is the twist axis or a-axis. A twist and a bend may be applied at the same location without additional limitations beyond those present for twisting or bending alone, except where the material properties of the fixation plate are excessively altered by the strain induced by the deformation.

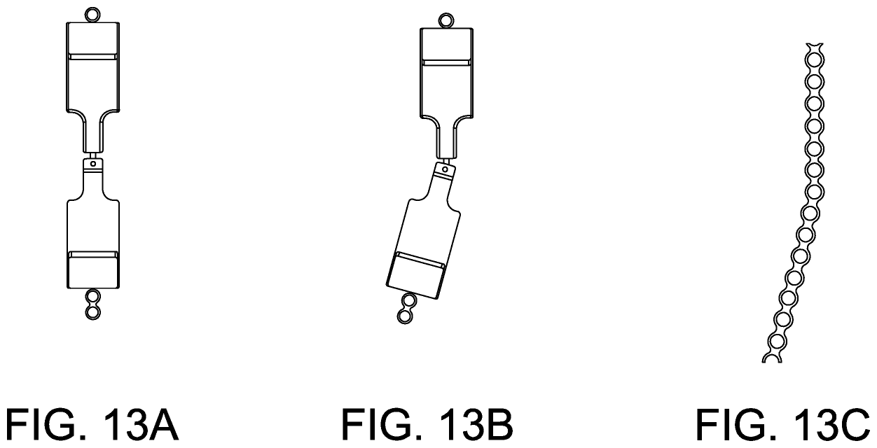

In FIGS. 13A-13C, the general operation for in-plane bending of the fixation plate is shown (e.g., a yaw operation or bending about the c-axis). The center of rotation is about the midpoint between the two gripped eyelets (e.g., the central bending point 104). Reset and indexing to the next eyelet is identical to that shown in FIGS. 11A-11I, with the exception that the operation in FIG. 11F would see the jaws rotating back to the origin along a different axis, that is the axis of the in-plane bend. Out-of-plane bending (e.g., FIG. 10) and twisting (e.g., FIGS. 12A-12C) may be combined with the technique for in-plane bending shown in FIGS. 13A-13C to produce three-dimensional manipulation of the eyelet normal vectors between any two adjacent eyelets. In FIGS. 13A-13C, a beveled nose is indicated on the opposing front surfaces of the jaw pairs. This beveled surface allows for a combination of in-plane, out-of-plane, and twisting motions without conflicts between the jaw pairs.

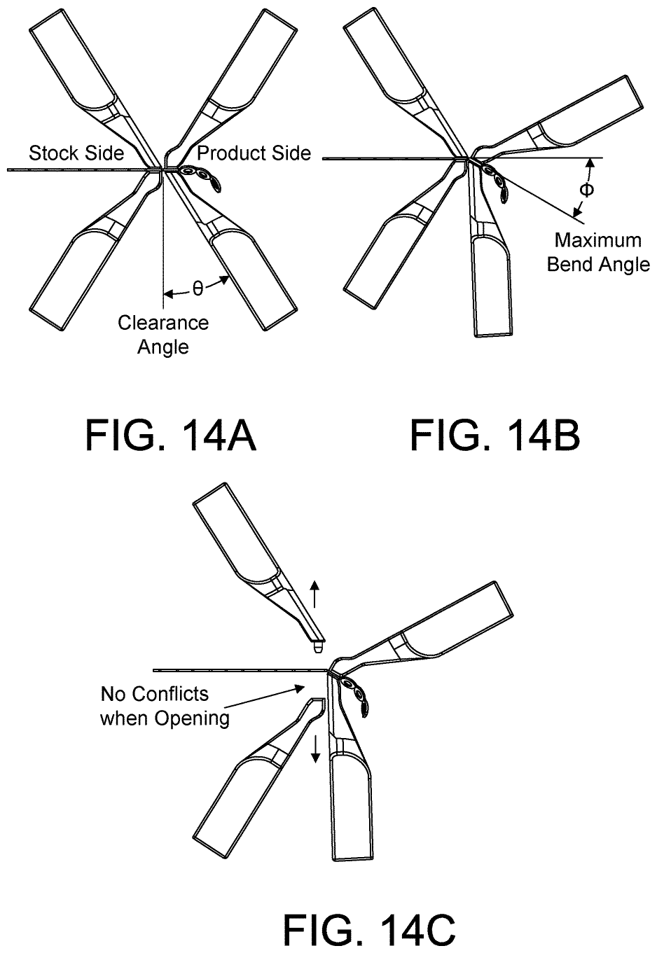

FIGS. 14A-14C show various angles of the members of the pairs of jaws of an example device to demonstrate the clearance angle concept. In FIG. 14A, a clearance angle (θ) is indicated. The clearance angle 74 must be greater than or equal to the maximum bend angle, om shown in FIG. 14B. This clearance angle is needed to allow the stock side jaws (left) to open without conflicts when the product side jaws (right) have bent the plate to the maximum bend angle φ, as shown in FIG. 14C.

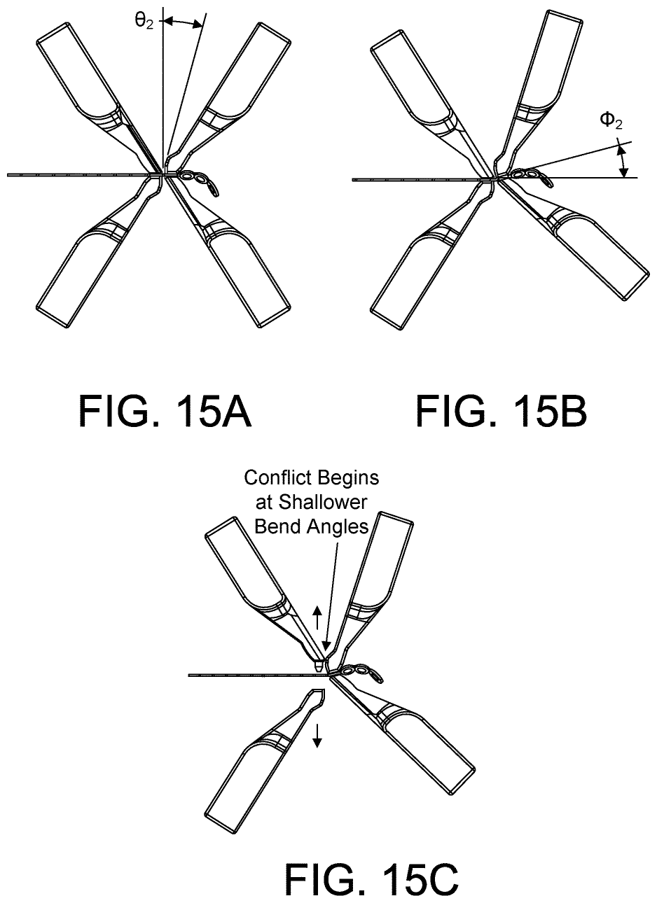

FIGS. 15A-15C show another implementation of the disclosed device to demonstrate the clamping jaw thickness concept. As mentioned previously, in some implementations of the device, the structure of the clamping jaw may be thicker near the bend site such that there may be a reduction in the maximum out-of-plane bending angle. In FIG. 15A, a device is shown having two opposing pairs of jaws (e.g., similar to the device 100). The device is shown applying a counterclockwise bend to the bone fixation plate (e.g., a counterclockwise pitch rotation). When applying the counterclockwise bend, a conflict between the stock side indexing jaw (left) and the product side clamping jaw (right) arises at a shallower bend angle when the stock side jaws are opened. This conflict occurs earlier only because of the thickened structure of the clamping jaw on the product side. However, this disclosure contemplates a variety of geometries of the device and associated jaws such that the conflict shown does not occur.

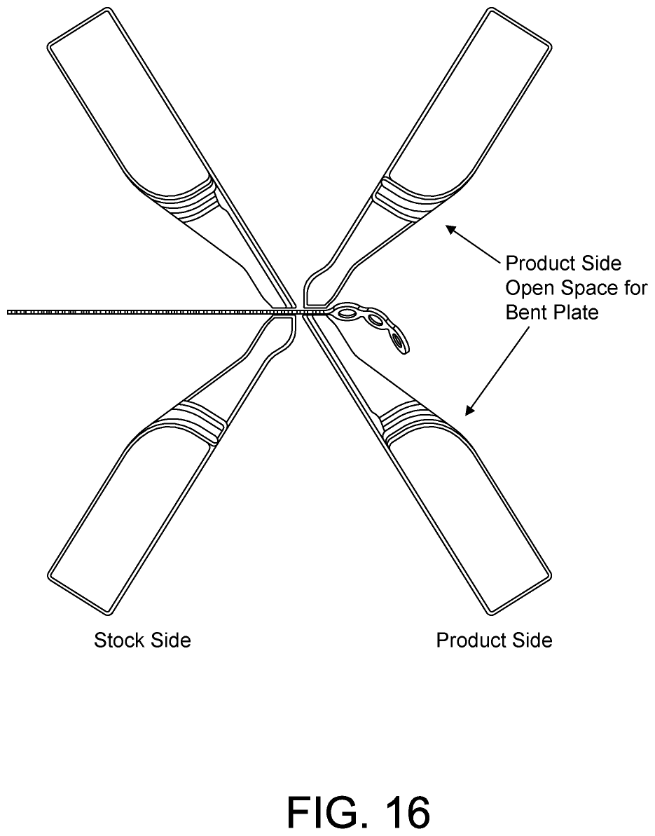

FIG. 16 illustrates the advantage of the space on the product side of the device (e.g., the second clearance area 132 between the third member 122 and the fourth member 124). The geometry of the back side (right side) of the product side jaws is chosen to allow space for the bent and twisted plate to exist without conflicting with the structures of the jaws. A similar space is present on the stock side jaws (e.g., first clearance area 130 between the first member 112 and the second member 114) to permit the presence of a bent plate if it becomes necessary to index back to a previously bent section of the plate. An open space outside of both jaw pairs is also used in some implementations when working with branching fixation plates, or with other complex plate geometries requiring clearance.

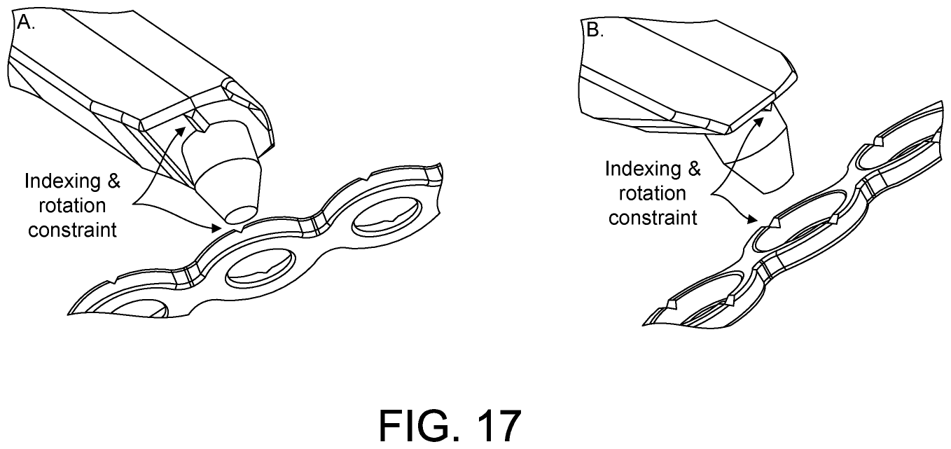

FIG. 17 shows an implementation of an indexing member (e.g., a tip of a member of a pair of jaws that includes one or more protrusions). In FIG. 17, the tip of the jaw shown includes an additional indexing feature. Additionally, a corresponding notched feature is included in the fixation plate. This feature also provides a rotation constraint between the jaw and the fixation plate which would aid in the operation of the in-plane bending (e.g., yaw rotation). This geometry may take the form of a wedge, half cylinder, or spherical bump, alone or in combination, with matching negative features in the fixation plate.

FIG. 18 shows another implementation of an indexing member (e.g., a tip of a member of a pair of jaws that includes one or more protrusions). In FIG. 18, an indexing feature is shown which may be applied to any jaw which requires a matching feature to be present in the fixation plate. This type of indexing feature is suitable for areas of fixation plates where holes are not present/desirable. An indexing tooth may also be used in addition to this indexing feature. This geometry may take the form of a wedge, half cylinder, or spherical bump, alone or in combination, with matching negative features in the fixation plate.

FIG. 19 shows another implementation of an indexing member (e.g., a tip of a member of a pair of jaws that includes one or more protrusions). The alternative indexing geometry of FIG. 19 wraps around the outside of some aspect of the fixation plate to provide accurate indexing. In this case, the outer diameter of the eyelet is used, but other geometry may also be used for indexing. This type of indexing feature may be used in conjunction with an indexing tooth, where the indexing tooth is present on the opposing jaw or the same jaw as the wrap-around indexing feature, but not on both. Similarly, this wrap-around geometry is also compatible with the indexing and rotation constraining features shown in FIGS. 17 and 18, where such features may be used on one or both clamping jaws where one jaw possesses the wrap-around geometry. This wrap-around geometry can also be designed to constrain the rotation of the plate.

The devices for plate bending disclosed herein (e.g., the devices shown and described in FIGS. 8-19) are contemplated to include an outer manufacturing frame and structure to support each of the pair of opposing jaws. For example, the devices may be disposed on a tabletop or larger device having an outer frame supporting one or more pairs of jaws and being rotatable via one or more actuators. This larger framework of the disclosed device (e.g., a system including the disclosed device) may further include actuators (e.g., motors, pneumatics, hydraulics, etc.), controllers, sliding rails, a user interface, complicated frame and bearing systems to accommodate rotation of one or both halves of the machine relative to one another at once.

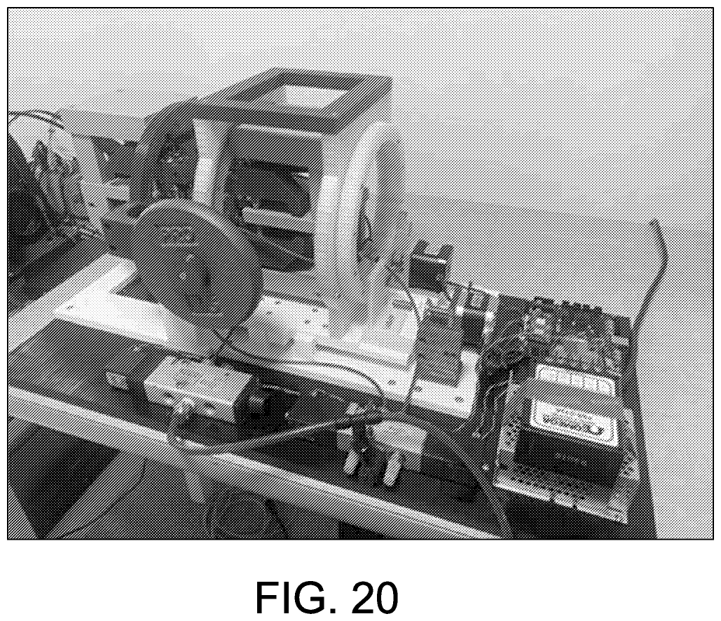

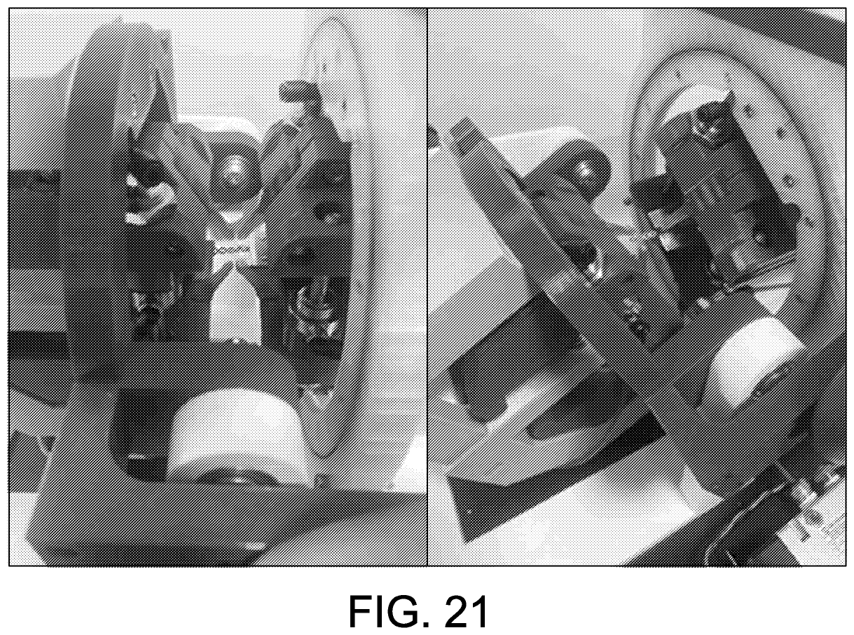

FIGS. 20 and 21 show one implementation of a system including the disclosed device along with associated framing, actuators, and controllers to facilitate rotation of the opposing jaws. Specifically, FIG. 20 and FIG. 21 show images of a successfully demonstrated fixation plate bending machine utilizing the jaw designs of this disclosure. The device makes use of the plate-bending process described herein for both out-of-plane bending and twisting. The device is able to automatically index between adjacent pairs of eyelets to give out-of-plane bends/twists to every eyelet pair according to a software defined sequence of bends derived from the surface of a 3D model. The out-of-plane bending range is +30 to −15 degrees (one bend direction is limited by the thickening of the clamping jaw, see FIG. 9). The twisting range is limited only by the plate material properties.

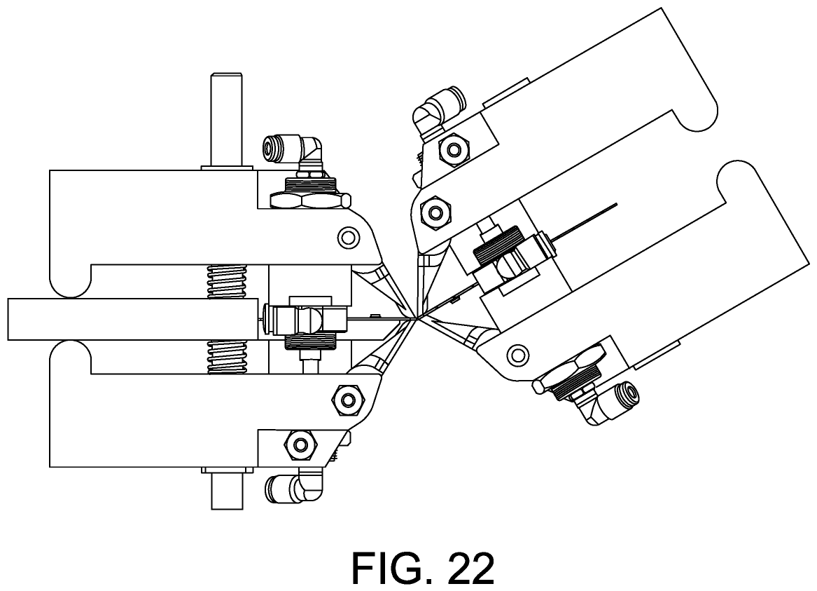

FIGS. 22-24 show another implementation of a fixation plate bending machine (e.g., a system including the devices disclosed herein). The system of FIGS. 22-24 can perform out-of-plane bending, twisting, and in-plane bending (e.g., all of the pitch, roll, and yaw operations). The frame of the system includes offset/angled surfaces, similar to the members of the opposing jaws, to reduce the interference possibility and increase the maximum bend angle. The device includes pneumatic systems for moving and applying pressure with the jaws. Electric actuators may also be used in place of the pneumatic system.

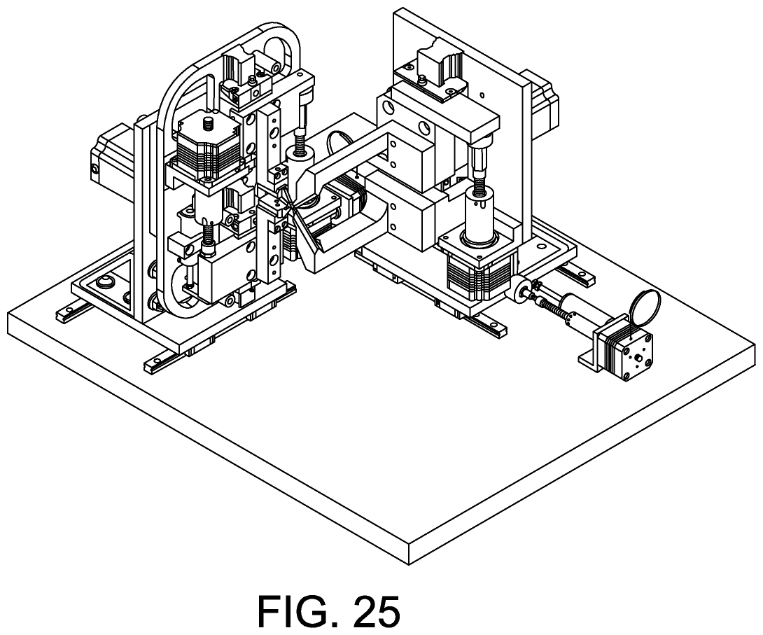

FIGS. 25-29 show another implementation of a fixation plate bending machine (e.g., including the devices disclosed herein). The system of FIGS. 25-29 can perform out-of-plane bending, twisting, and in-plane bending (e.g., all of the pitch, roll, and yaw operations). Each of the pairs of jaws is supported on an independent framework having a plurality of actuators configured to initiate rotation and/or translation. For example, an actuator is positioned to translate one of the pairs of jaws and the connected system architecture along a base rail system. Such a system may be useful when indexing to a branch of a non-linear bone fixation plate.

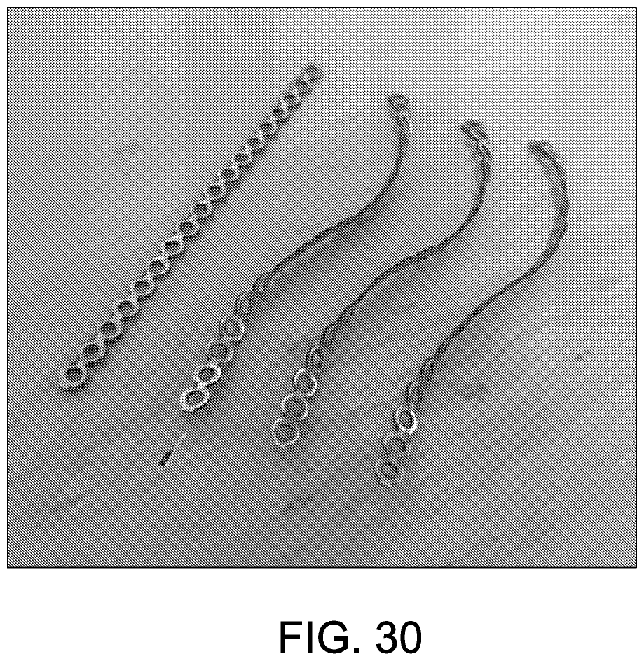

FIG. 30 shows images of finished bone fixation plates formed by the devices of this disclosure. Specifically, one unbent and three bent fixation plate analogs are shown. Each was bent according to the same sequence of bends/twists in the machine depicted in FIGS. 20-21.

The machine was able to fully bend the plates within one minute and fifteen seconds. The analog plates have 18 eyelets and received 17 bend/twist combinations from the machine of various angles requested by the software which generated the required plate geometry.

### Example Implementation and Advantages of the Disclosed Systems, Methods, and Devices

One example implementation of the disclosed process and device is disclosed along with a discussion concerning advantages, alternatives, and optional considerations. For example, the process of scanning a patient's anatomy, producing a model of a medical implant based on the scan, and delivering instructions to produce the medical implant to a device are each described. The particular example shown is related to the skull (e.g., mandible) of a patient; however, other anatomical locations are contemplated by this example.

### Significance of the Disclosed Workflow

Reconstructive skeletal surgeries are often simulated virtually (i.e., on a computer) to obtain the target shape of a site to be reconstructed and to identify sites for fixation hardware placement. However, these surgical simulation activities do not provide the surgeon with biomechanical properties of the post-operative anatomy (i.e., functional outcome) nor do they inform the surgeon's choice of fixation hardware geometry, material, or location.

In many cases the virtually reconstructed bone is 3D printed and fixation plates are bent to those models to provide optimal fit. It is known that reducing the gap, so that the plate is flush to the bone, increases fixation stability. However, beyond flush plating, predicting outcomes is left to the surgeon's experience and medical judgment. Today's personalized CMF (craniomaxillofacial) reconstructive surgery can involve more art (of medicine) than (data-driven) science. If biomechanical data were available, it could be used by the surgeon to choose the optimal fixation plate shape, thickness, length, footprint, and location, as well as the fixation plate screw location, type, and length. All of these variables play a biomechanical role in bone healing as well as the healed bone's subsequent ability to fully restore oral function. If available, a 3D-printed biomimetic model that captured an optimal reconstructive surgery plan could be used to validate a biomechanical analysis made by physicians and engineers who use virtual surgical planning (VSP) software. One deterrent to VSP and biomechanical analysis is the lengthy preparation required for headquarter-based manufacturers to follow HIPAA requirements to obtain a patient's 3D CT (Computed Tomography) scan, prepare a VSP and personalized fixation plan, obtain physician approval, and fabricate and ship the approved device. Indeed, many large, tertiary care medical centers do not currently utilize any of these services.

Because there is virtually no biomechanical aspect to the planning process, high levels of mechanical failure are considered “acceptable”. Failures may be attributed to medical experience and judgment or the use of overly stiff materials that cause stress shielding and bone resorption, implant loosening, device failure, and revision surgery. However, the lack of biomechanical data makes it impossible to avoid stress shielding or, another possibility, stress concentration. Stress concentration, especially in work-hardened “crimp” (i.e., thinned areas for bending) zones, may lead to device failure.

Indeed, 36-39% of hemi-mandibular graft fixation devices can be expected to fail and require revision surgery, with 8-10% of all CMF fixation plates having been observed to break, loosen, or in other ways fail during normal activities. In addition to the painful emergency caused by unexpected failure of these devices, typical re-operation costs average $50,000. While it usually takes longer to manifest than CMF fixation failure, stress shielding of hip implants commonly results in aseptic loosening. To avoid this, there have been attempts at modulating mechanical properties using multi-material devices. In cases of cancer or trauma, having these resources available at the point-of-care would facilitate personalization without delaying reconstructive surgery. By providing CMF surgeons with interactive biomechanical data in point-of-care VSP, the surgeons are allowed to choose a graft fixation device's geometry, material, and location so as to avoid bone stress-shielding and device-stress concentration, thereby optimizing patient outcomes.

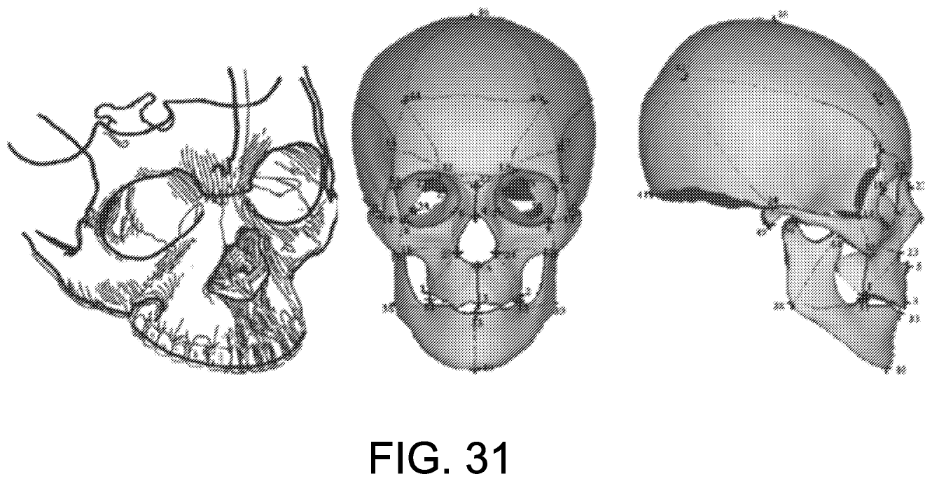

The disclosed Virtual Surgical Planning (VSP) would begin with an established map of CMF 3D CT-imaged skull surface anatomy that locates biologically homologous features in regions of high surface curvature. See FIG. 31 showing surface curvature-mapped 3D CT images, which are the source of crestline (solid) and geodesic (dotted) line wireframe skull templates. The skull template was fit to new patient images by matching high curvature anatomical landmarks. The average surface images have a grid of points on each surface tile and a tetrahedral mesh for the skull which, along with the CT density data, are used to set up a biomechanical model of the skull.

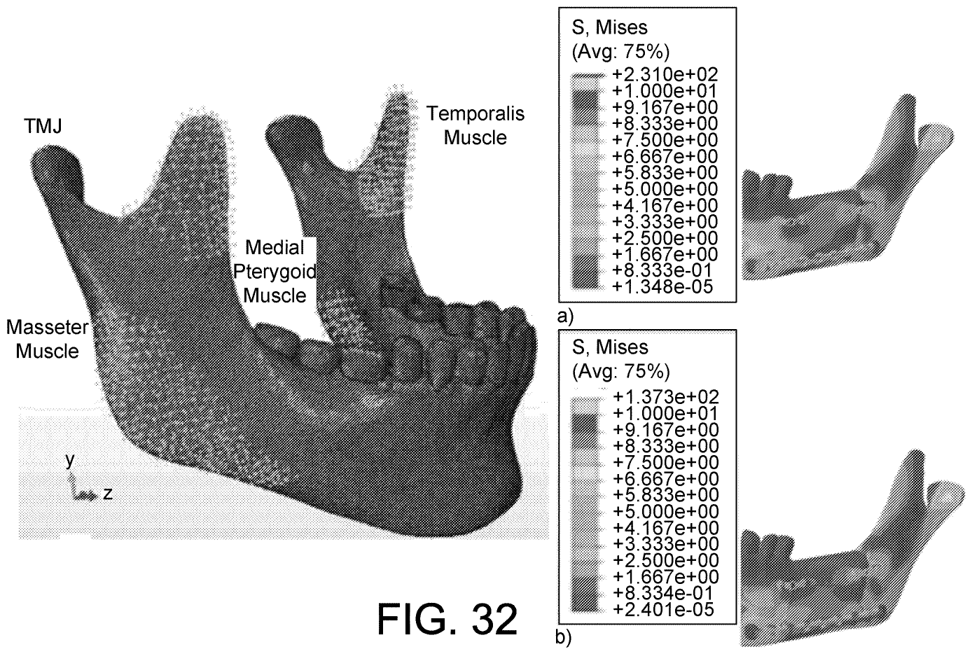

This homology map is then used to superimpose a normative biomechanical model of chewing to a patient's 3D CT-based image (e.g., FIG. 32). Specifically, FIG. 32 shows muscle force vectors and stiffness matching wherein muscle vectors and force databases (left) are used to determine a patient's muscle power from the muscle's maximum cross-sectional area. Stiffness-matched NiTi graft fixation at the bottom-right shows less stress concentration than Ti-6A1-4V fixation at the upper right. The POC (Point-of-Care) VSP surface template (e.g., FIG. 31) breaks down the patient's 3D CT surface image into homologous landmarks, crestlines, and tiles, thus mapping the shape found in all normal skulls. Once mapped, osteotomies can be performed in VSP software, and 3D CT-imaged bone grafts are mapped and placed to fill segmental defects (full gaps). The material properties of 3D CT-visualized bone can be readily determined, allowing 3D-printed biomimetic models of the skull. Furthermore, CMF fixation devices may be formed manually, by a robot (e.g., the disclosed device of FIG. 8), or by 3D printing. Preoperative forming of fixation plates saves OR time otherwise required to manually bend them.

A virtual surgical planning (VSP) biomechanical model that includes normal bone, bone grafts, fixation, and chewing forces would aid in the selection and location of fixation hardware to be personalized (bent) and provide the surgeon with data that should help avoid harmful stress shielding of the bone and stress concentrations in the device. Current surgical simulation software is silent in terms of information that can help the surgeon choose a fixation strategy from among FDA-approved devices. The surface map described above in FIG. 31 maps the outer surface of the skull, providing a grid of regularly spaced surface landmarks that can be used to create a tetrahedral mesh with valid mechanical properties (i.e., finite element model). The assigned mechanical properties of the skull can be directly derived from 3D CT data (e.g., FIG. 32). The mechanical model of chewing is seen in FIG. 32 is derived from personalized muscle vectors (i.e., the direction of pull) and force magnitude (i.e., chewing strength) calibrated by its relationship to the maximum cross-sectional area of each muscle. Based on expected loading, the finite element mesh is assigned mechanical properties, and a loading model is initially generated from average biomechanical templates. The finite element model is personalized with that person's muscle vectors (i.e., the direction of pull) and force magnitudes (i.e., chewing strength).

The spectrum of fixation options varies between relatively thin, simple dog-bone-shaped mini plates, to L, U, square, or complex shapes, including thick reconstruction bars that can be bent and cut to fit over mandibular or maxillary grafts. The screw holes may accept locking screws (i.e., threads on the screw head). However, it would be helpful if one could model normal stress-strain trajectories and confirm they are uninterrupted by CMF fixation stress concentrations. Stress concentrations may occur in thin areas of a fixation device or where there is a gap between the plate and the bone surface. Surgeons currently rely on experience and medical judgment, not empirical data, when selecting CMF fixation devices. That selection also depends on the shape of the underlying bone. Indeed, 3D printed CMF fixation of some examples emphasizes contact surface area. If healing is at risk from chewing forces, additional immobilization from maxillomandibular fixation (MMF) may be used. Assuming a good fit, a biomechanical model relating bite force to hardware selection will dramatically improve outcomes.

### Point-of-Care Manufacturing

Hybrid Autonomous Manufacturing (HAM) robotic bending of CMF fixation for closed-loop fabrication is cost-effective, would save time, increase plate bending precision, and decrease the rate of plate failure. Where available, 3D-printed VSP models of a desired CMF surgical reconstruction are often used as a target substrate for pre-operative, manual fixation hardware bending. Models often provide better access to the surface of interest than the surgical window. However, with major reconstructive surgery, a surgeon may spend hours bending plates to a VSP model. Only at the most advanced tertiary care centers, are personalized CMF fixation devices 3D printed locally. A proposed project would compare all three plating strategies: (1) manually-bent, (2) 3D printed, and (3) robotically-bent fixation plates. Reducing surgical time also reduces risk and procedure costs for the patient. Compared to manual bending, the disclosed robotic bending system, unlike previous attempts, uses (a) VSP to guarantee a flush surface fit, (b) process engineering for a stepwise approach, and (c) uniform bending and machining to reduce localized work hardening (e.g., at thinned crimp points) that risks subsequent fatigue failure.

Closed loop fabrication (i.e., feedback between design, fabrication, and functional outcome) is made possible by Hybrid Autonomous Manufacturing (HAM) (e.g., see FIG. 1). Unlike stamping or manual or robotic bending with two grip points, the proposed study would allow optimized fabrication work paths that most efficiently reach the intended shape while maintaining desired mechanical properties. For robotic fixation plate bending, knowing the shape and mechanical properties of commercially available fixation plates would allow for the creation of design envelopes for Quality Management Systems (QMS) that would ensure reliability. Point-of-Care Manufacturing (POCM) should inform the optimization of future fixation plate designs for robotic bending.

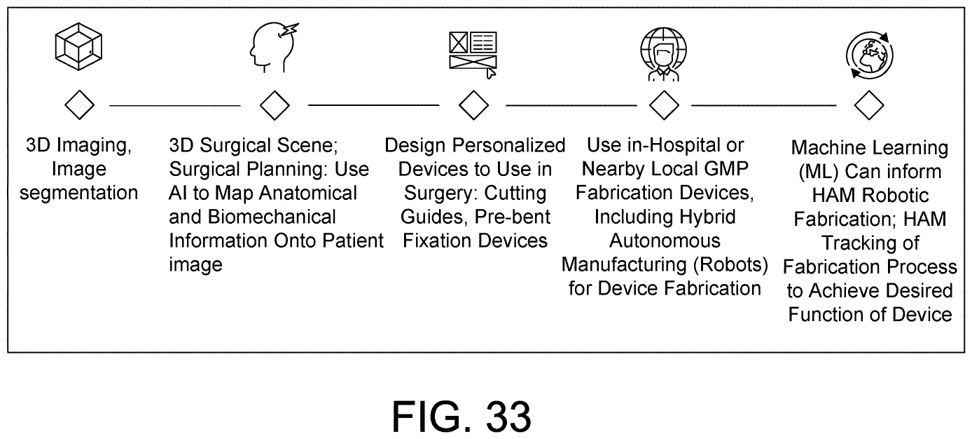

Companies could anonymously capture best practices data from POCM sites using their software-see the POCM design envelope showing an optimized fabrication and device function in FIG. 33. Distributed medical device manufacturing would incentivize establishing or recruiting companies located near hospitals with Good Manufacturing Practices (GMP) for tasks such as plate forging, drilling, and robotic bending.

Biomimetic models could be used to develop new procedures and test device “use-life”, mock complex surgeries, facilitate proctored training of new techniques, and validate new materials, fabrication procedures, or devices. Biomimetic CMF models could also: (a) reduce the extent that large animal or cadaveric tissue testing would be needed for research into new therapies, (b) pre-operative mock testing of complex craniofacial procedures, (c) provide a model with the plates already bent and applied to surgeons in the operating room to show the intended surgical plan, or (d) a biomimetic model to certify POCM activities.

Point-of-Care Manufacturing of CMF fixation would improve outcomes for patients with emergent conditions (e.g., cancer, trauma) who cannot wait for centralized production site design and fabrication. There is urgency in cases with invasive CMF tumor or significant craniofacial trauma that may not be compatible with timelines for the current corporate, headquarter-centralized, design and manufacture of personalized CMF fixation. Assuming a smooth path for HIPAA protected data from the hospital to the company is established, days may be lost due to differences in schedules and time zones between surgeons and engineers, shipping of finished osteotomy guides and fixation devices, and the receiving hospital's sterilization of those devices. With CMF trauma, stabilizing and definitive surgery within a 2-day window is associated with the best outcomes, while a delay of 1-2 weeks is associated with a reduced ability to heal and restore normal function. Similarly, the CMF cancer “treatment package” time is focused on time from diagnosis to post-surgical wound healing to follow-on radiation therapy. Wound healing needs to be established before radiation treatments occur, as radiation will slow that process. Each day lost preparing for surgery has an effect that may not be tolerable. For example, in a large recent CMF study, average overall survival was found to be 2.5 years less when starting radiation therapy 7 weeks post-diagnosis versus 6 weeks. POCM of CMF fixation stands to improve outcomes through earlier healing, restored function, and reduced morbidity and mortality, especially in cases of trauma and cancer.

Why biomechanically-informed POCM is the optimal solution for CMF fixation: (1) Established curvature-mapping tools can be used to superimpose a biomechanical model that would allow surgeons to make an informed selection from available fixation devices based on mechanical models of restored function and will set a quantitative functional baseline to compare with actual outcomes (e.g., reduced re-operation); (2) VSP biomechanical information would allow for comparing outcome of manual plate bending to a 3D printed surgical model versus robotic fixation plate bending with mechanical property tracking; (3) multiple plates can be bent simultaneously by more than one robot; (4) anonymous feedback to software vendors from users can assist in making future improvements (best practice) to fabrication process planning software; (5) distributing manufacturing to the Point-of-Care will shorten production times, reduce costs, and make personalized fixation available to patients with time-sensitive emergent conditions (e.g., cancer, trauma).

### Workflow Design and Experimental Methods

Surgical planning can map CMF biomechanics to inform the choice of shape, material, location, and robotic, Point-of-Care Manufacturing (POCM) toolpath for graft fixation plates. Optimizing CMF fixation size and shape can be informed by a personalized biomechanical model of reconstructive surgical outcomes for maximum/likely bite force.

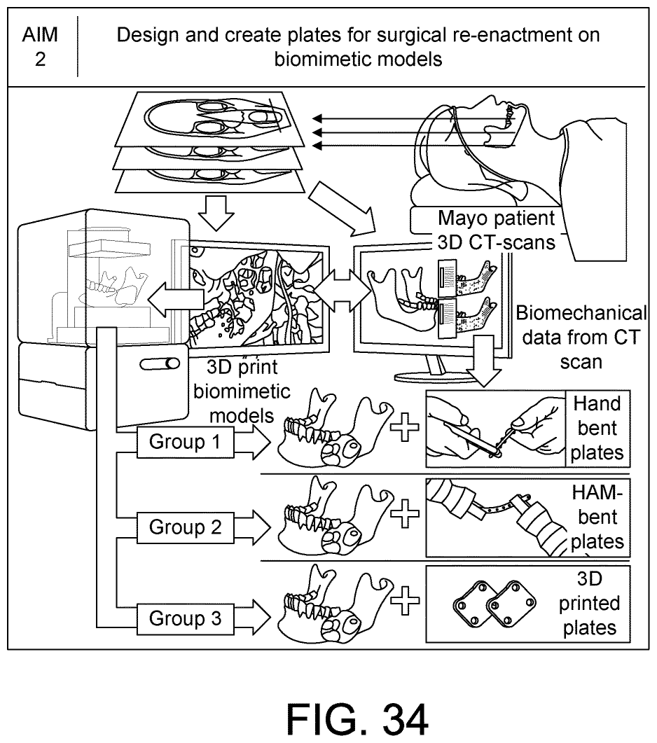

A novel surgical simulation system disclosed herein may be used to merge both shape and structural (mechanical) properties derived from patient 3D CT images in order to design optimal mandibular bone grafts and graft cutting guides and to select and bend graft fixation hardware. For example, see FIG. 34 showing a diagram of the design and creation of plates for surgical re-enactment on biomimetic models. While both shape and biomechanical data have been previously collected, this method uses established surface curvature-based homology maps of patient skull images to apply a normative deformable template that will include an established biomechanical model of chewing. The biomechanical model begins with mapping (labeling) a tetrahedral mesh of cranial and bone graft anatomy that can be used in a biomechanical model of chewing both before and after reconstructive surgery. Currently, CMF surgeons have virtually no biomechanical information from which to base their choice of fixation device, the location where it is best placed, how it will interact with the bone to which it is attached, the muscles that will move that bone, or the amount of personalization (i.e., fixation geometry, location, materials, and bending) that will be needed. Surgeons guiding the osteotomies in the surgical plan would use an interactive environment that will show the biomechanical outcome of their chosen reconstruction plan. The surgeon will use this information to obtain optimal post-operative restoration of normal chewing function. Finally, that model of chewing will inform the selection, location, and pre-operative personalization (i.e., form-fitting fabrication) of the fixation plates.

Key Example Method: First, the patient's 3D CT image is segmented (i.e., 3D bone surface identified) and curvature mapped in Amira (Fisher Scientific, Waltham, MA). The curvature map allows the superimposition of a well-studied deformable template to the 3D skull surface image. Muscle vectors and forces are determined based on the maximal cross-sectional area of the three masticatory muscles for the simulated restored (post-surgical) anatomy during VSP in Geomagic. Unbent fixation devices prepared in SolidWorks (Dassault Systèmes, Vélizy-Villacoublay, France) and nTopology (New York, NY), are sent as tessellated surfaces to the VSP (i.e., Geomagic Freeform) to be used both in choosing optimal attachment location and biomechanical performance, once screw placement has been planned, under maximum chewing force regime by exporting the scene from HyperMesh 3D (i.e., creating a 3D mesh) to ABAQUS (Dassault Systèmes), COMSOL (Stockholm, Sweden), or Ansys (Canonsburg, PA) for Finite Element Analysis (FEA) for optimization of fixation geometry and bone mooring location.

Fixation shape, location, and screw location and length are studied under cyclic and traumatic loading to determine the stiffness of the reconstructed anatomy and to ensure that personalized fixation hardware does not stress-shield bone or cause stress concentrations in the fixation hardware. This simulation ensures that the grafted bone and fixation device do not interrupt normal masticatory stress/strain trajectories and that the surgical reconstructive hardware will not fail during healing.

The study may further optimize Hybrid Autonomous Manufacturing (HAM) (robotic forming) of fixation plates via global-deformation (e.g., see FIG. 2 and the workflow described therein). That will bring about fast, accurate, and reproducible forming of CMF fixation plates via a closed loop and spring-back corrected forming for dimensional and mechanical performance accuracy. The study further provides for mechanical testing of biomimetic surgical mock-ups manufacturing tolerances on final fixation plate geometry and biomechanics. Both are dependent on the variability in the CMF fixation plates. The study will utilize a non-intrusive uncertainty modeling framework to model the expected variability in final part geometry and its impact on the performance of the device with respect to cyclic and traumatic loading.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A bending device comprising:
a first pair of opposing jaws on a first end of the device including a first member disposed on a first side of a bending area and a corresponding second member disposed on a second side of the bending area opposite of the first side, wherein the first and second members extend away from the bending area at an angle such that a first clearance area is defined between the first and second members, wherein a first tip of the first member is configured to engage with a second tip of the second member within the bending area;
a second pair of opposing jaws on a second end of the device opposite the first end, the second pair of opposing jaws including a third member disposed on the first side of the bending area and a corresponding fourth member disposed on the second side of the bending area opposite of the first side, wherein the third and fourth members extend away from the bending area at an angle such that a second clearance area is defined between the third and fourth members, wherein a third tip of the third member is configured to engage with a fourth tip of the fourth member within the bending area; and
at least one actuator configured to move the first pair of jaws between (i) an engaged configuration wherein the first tip engages the second tip and (ii) a separated configuration wherein the first tip is separated from the second tip by a distance, the at least one actuator further configured to rotate the second pair of jaws to an angled configuration with respect to the bending area.

2. The bending device of claim 1, wherein the at least one actuator is further configured to move the second pair of jaws between (i) an engaged configuration wherein the third tip engages the fourth tip and (ii) a separated configuration wherein the third tip is separated from the fourth tip by a distance, the at least one actuator further configured to rotate the first pair of jaws to the angled configuration.

3. The bending device of claim 1, wherein the first and third members extend away from the bending area such that a third clearance area is defined between the first and third members, and wherein the second and fourth members extend away from the bending area such that a fourth clearance area is defined between the second and fourth members.

4. The bending device of claim 3, wherein, when the second pair of jaws are moved to the angled configuration, the third member moves into the third clearance area closer to the first member.

5. The bending device of claim 3, wherein the third and fourth clearance areas ensure that the first pair of jaws and the second pair of jaws do not interfere with each other during operation of the bending device.

6. The bending device of claim 1, wherein rotation of the second pair of jaws to the angled configuration comprises one or more of: (i) rotation of the second pair of jaws about a first axis, defined as a roll rotation; (ii) rotation of the second pair of jaws about a second axis, defined as a pitch rotation; and (iii) rotation of the second pair of jaws about a third axis, defined as a yaw rotation.

7. The bending device of claim 1, wherein the bending area includes a center point of rotation defined in between each of the first, second, third, and fourth tips.

8. The bending device of claim 1, wherein the first tip of the first member of the first pair of opposing jaws comprises a protrusion and the second tip of the second member of the first pair of opposing jaws comprises a cavity, wherein the protrusion engages the cavity when the first pair of opposing jaws is in the engaged configuration.

9. The bending device of claim 1, further comprising:
a controller in communication with and configured to activate the at least one actuator to move one or more of the first pair of opposing jaws and the second pair of opposing jaws.

10. The bending device of claim 9, wherein the controller comprises a set of manufacturing process instructions for moving the first and second pair of opposing jaws in a predefined sequence.

11. The bending device of claim 10, wherein the instructions are based on a computational model of a patient's anatomy or a model of an implant.

12. The bending device of claim 1, wherein the second pair of opposing jaws is configured to bend a bone fixation plate to a desired angle or an angled configuration.

13. A system comprising:
a bending device comprising:
a first pair of opposing jaws including a first member disposed on a first side of a bending area and a corresponding second member disposed on a second side of the bending area opposite of the first side, wherein the first and second members extend away from the bending area at an angle such that a first clearance area is defined between the first and second members, wherein a first tip of the first member is configured to engage with a second tip of the second member within the bending area; and
a second pair of opposing jaws including a third member disposed on the first side of the bending area and a corresponding fourth member disposed on the second side of the bending area opposite of the first side, wherein the third and fourth members extend away from the bending area at an angle such that a second clearance area is defined between the third and fourth members, wherein a third tip of the third member is configured to engage with a fourth tip of the fourth member within the bending area;
at least one actuator coupled to the bending device, the at least one actuator configured to move the first pair of jaws between (i) an engage configuration wherein the first tip engages the second tip and (ii) a separated configuration wherein the first tip is separated from the second tip by a distance, the at least one actuator further configured to rotate the second pair of jaws to an angled configuration with respect to the bending area;
a medical implant disposed within the bending area, wherein each of the first pair of opposing jaws and the second pair of opposing jaws are configured to engage with the medical implant; and
a control system in communication with the bending device, the control system comprising computer readable instructions for bending the medical implant to a desired shape, the computer readable instructions being based on an anatomical model of a patient.

14. The system of claim 13, wherein the medical implant is a bone fixation plate.

15. The system of claim 14, wherein the bone fixation plate includes a plurality of eyelets and wherein the first and second tip of the first pair of jaws are used to index or locate the bone fixation plate via the plurality of eyelets.

16. A method of shaping a medical implant, the method comprising:
providing a device comprising a first pair of opposing jaws on an input side of a bending area and a second pair of opposing jaws on an output side of a bending area, wherein each of the first and second pair of opposing jaws further define one or more clearance zones to prevent interference between the first and second pairs of opposing jaws;
moving the medical implant into the bending area between the first pair of opposing jaws from the input side;
closing, via at least one actuator, the first pair of opposing jaws to engage the medical implant at a first portion of the medical implant;
closing, via the at least one actuator, the second pair of opposing jaws to engage the medical implant at a second portion of the medical implant; and
turning, via the least one actuator, the second pair of opposing jaws to a first angled configuration with respect to the bending area such that the second portion of the medical implant is angled with respect to the first portion of the medical implant.

17. The method of claim 16, further comprising:
opening, via the at least one actuator, the first pair of opposing jaws; and
moving the first pair of opposing jaws to a third portion of the medical implant that is closer to the input side than the first portion of the medical implant and closing the first pair of opposing jaws to engage the third portion of the medical implant.

18. The method of claim 17, further comprising:
opening, via the at least one actuator, the second pair of opposing jaws; and
moving the second pair of opposing jaws to the second portion of the medical implant and closing the second pair of opposing jaws to engage the third portion of the medical implant.

19. The method of claim 16, wherein the medical implant is shaped into a custom topology via one or more steps of turning the second pair of opposing jaws to one or more angled configurations, wherein the custom topology is based on a model of a patient's anatomy.

20. The method of claim 19, wherein, in each of the one or more steps of turning the second pair of opposing jaws, the medical implant experiences a single bending operation, reducing iterative bending of a single area that may produce fatigue.
